(19) United States
(12) Patent Application Publication
(10) Pub. No.: US 2024/0409505 A1
(43) Pub. Date: Dec. 12, 2024

(54) GLUCONAMIDE SULFATE SURFACTANTS

(71) Applicant: The Procter & Gamble Company, Cincinnati, OH (US)

(72) Inventors: Randall Thomas REILMAN, Cincinnati, OH (US); John August WOS, Mason, OH (US); Phillip Kyle VINSON, Fairfield, OH (US); Jan Julien Marie-Louise BILLIAUW, Gentbrugge (BE)

(21) Appl. No.: 18/735,741

(22) Filed: Jun. 6, 2024

Related U.S. Application Data
(60) Provisional application No. 63/472,385, filed on Jun. 12, 2023.

(51) Int. Cl. C07C 305/04; A61K 8/23; C07C 305/14; C07D 209/08; C11D 1/90

(52) U.S. Cl. CPC C07C 305/04; A61K 8/23; C07C 305/14; C07D 209/08; C11D 1/90

(57) ABSTRACT

Gluconamide sulfates that may be used in consumer products to provide surfactant properties.

### FIELD OF THE INVENTION

Gluconamide sugar sulfates, which may be used in consumer products to provide surfactant properties.

### BACKGROUND OF THE INVENTION

Surfactants are widely used in everyday life in the form of consumer product compositions, such as detergents and shampoos to remove soils and provide the cleaning indicator of suds generation. There are many kinds of surfactants, and depending on their charge states at physiological pH, are classified as anionic surfactants, cationic surfactants, nonionic surfactants, and amphoteric surfactants.

Alkyl and alkyl ether sulfates form, by far, the most popular group of anionic surfactants used as primary surfactants in shampoos. The alkyl and alkyl ether sulfates most widely used include sodium lauryl ether sulfate, sodium lauryl sulfate, ammonium lauryl ether sulfate, and ammonium lauryl sulfate. These are cost-effective materials which, if correctly formulated, deliver effective cleansing, foaming, rheology control, and polymer deposition; but they have a disadvantage in that while they provide excellent washing power, they can cause strong skin irritation or leave hair brittle. To improve these deficiencies, a technique of adding hydrophilic ethylene oxide during the production of sodium laureth sulfate or ammonium laureth sulfate was developed to provide surfactant with sudsing properties, but reduced irritation as compared to conventional sodium lauryl sulfate or ammonium lauryl sulfate.

Anionic surfactants have been used, typically in combination with cosurfactants, especially amphoteric and zwitterionic co-surfactants such as amine oxide and betaines, to provide suds during dishwashing, with alkyl sulphate and alkyl alkoxy sulphates found to be particularly effective at providing improved sudsing in addition to the desired cleaning.

Ethoxylated surfactants are currently an anionic surfactant class heavily used for these purposes.

Accordingly, in order to solve the above problems, the present invention provides a surfactant prepared without the use of ethoxylation.

### SUMMARY OF THE INVENTION

The present invention relates to a compound comprising the following structure:

A compound comprising the following structure:

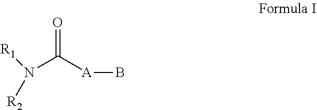

where:
   A=polyhydroxylated alkyl, saccharide;
   B=—OSO₃⁻M⁺, where is M is a cation that forms an acceptable salt;
   R₁=a heterocyclic, substituted or unsubstituted straight chain or branched chain heteroalkyl, alkyl or alkenyl, having 1 to 22 carbon and/or heteroatoms;
   R2═H; alkyl;
including all stereoisomers thereof.

In some embodiments, R₁ has an average alkyl chain length of from 8 to 18, preferably from 10 to 14, more preferably from 12 to 14, most preferably from 12 to 13 carbon atoms. In preferred embodiments R₁ is straight chain alkyl. In more preferred embodiments R₁ is straight chain alkyl that is bio-based.

In some embodiments, M is an alkali metal, an alkaline earth metal, ammonium, alkylammonium, or a mixture thereof. In embodiments M is an alkali metal, for example sodium.

A detergent composition is provided that comprises a compound having the following structure:

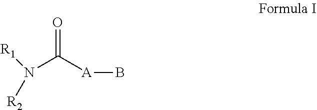

where:
   A=polyhydroxylated alkyl, saccharide;
   B=—OSO₃⁻M⁺, where is M is a cation that forms an acceptable salt;
   R₁=a heterocyclic, substituted or unsubstituted straight chain or branched chain heteroalkyl, alkyl or alkenyl, having 1 to 22 carbon and/or heteroatoms;
   R2═H; alkyl;
including all stereoisomers thereof.

In some embodiments, R₁ has an average alkyl chain length of from 8 to 18, preferably from 10 to 14, more preferably from 12 to 14, most preferably from 12 to 13 carbon atoms. In preferred embodiments R₁ is straight chain alkyl. In more preferred embodiments R₁ is straight chain alkyl that is bio-based.

In some embodiments, M is an alkali metal, an alkaline earth metal, ammonium, alkylammonium, or a mixture thereof. In embodiments M is an alkali metal, for example sodium.

### DETAILED DESCRIPTION OF THE INVENTION

The present invention describes a series of gluconamide sulfates (GS) that can be used as replacements for existing ethoxylated sulfate surfactants. The GS's of the present invention may be used in liquid detergent compositions to provide a more consistent sudsing experience, regardless of the hardness of the water used to make the wash solution. GS's when used in liquid detergent compositions also provide the compositions with a good sudsing profile, including high initial suds volume generation and sustained suds stabilization through a dishwashing process, even when in presence of greasy and/or particulate soils, as well as good grease removal.

### Definitions

As used herein, articles such as “a” and “an” when used in a claim, are understood to mean one or more of what is claimed or described.

As used herein, the word “or” when used as a connector of two or more elements is meant to include the elements individually and in combination; for example X or Y, means X or Y or both.

The term “comprising” as used herein means that steps and ingredients other than those specifically mentioned can be added. This term encompasses the terms “consisting of” and “consisting essentially of.” The compositions of the present invention can comprise, consist of, and consist essentially of the essential elements and limitations of the invention described herein, as well as any of the additional or optional ingredients, components, steps, or limitations described herein.

The term “dishware” as used herein includes cookware and tableware made from, by non-limiting examples, ceramic, china, metal, glass, plastic (e.g., polyethylene, polypropylene, polystyrene, etc.) and wood.

The term “grease” or “greasy” as used herein means materials comprising at least in part (i.e., at least 0.5 wt % by weight of the grease) saturated and unsaturated fats and oils, preferably oils and fats derived from animal sources such as beef, pig and/or chicken.

The terms “include”, “includes” and “including” are meant to be non-limiting.

The term “particulate soils” as used herein means inorganic and especially organic, solid soil particles, especially food particles, such as for non-limiting examples: finely divided elemental carbon, baked grease particles, and meat particles.

The term “sudsing profile” as used herein refers to the properties of a detergent composition relating to suds character during the dishwashing process. The term “sudsing profile” of a detergent composition includes suds volume generated upon dissolving and agitation, typically manual agitation, of the detergent composition in the aqueous washing solution, and the retention of the suds during the dishwashing process. Preferably, hand dishwashing detergent compositions characterized as having “good sudsing profile” tend to have high suds volume and/or sustained suds volume, particularly during a substantial portion of or for the entire manual dishwashing process. This is important as the consumer uses high suds as an indicator that sufficient detergent composition has been dosed. Moreover, the consumer also uses the sustained suds volume as an indicator that sufficient active cleaning ingredients (e.g., surfactants) are present, even towards the end of the dishwashing process. The consumer usually renews the washing solution when the sudsing subsides. Thus, a low sudsing detergent composition will tend to be replaced by the consumer more frequently than is necessary because of the low sudsing level.

The term “bio-based” material refers to a renewable material.

The term “renewable material” refers to a material that is produced from a renewable resource.

The term “renewable resource” refers to a resource that is produced via a natural process at a rate comparable to its rate of consumption (e.g., within a 100 year time frame). The resource can be replenished naturally, or via agricultural techniques. Non-limiting examples of renewable resources include plants (e.g., sugar cane, beets, corn, potatoes, citrus fruit, woody plants, lignocellulose, hemicellulose, cellulosic waste), animals, fish, bacteria, fungi, and forestry products. These resources can be naturally occurring, hybrids, or genetically engineered organisms. Natural resources, such as crude oil, coal, natural gas, and peat, which take longer than 100 years to form, are not considered renewable resources.

The term “bio-based content” refers to the amount of carbon from a renewable resource in a material as a percent of the weight (mass) of the total organic carbon in the material, as determined by ASTM D6866-10 Method B.

The term “petroleum-based” material refers to a material that is produced from fossil material, such as petroleum, natural gas, coal, etc.

The term “detergent composition” refers to a composition or formulation designed for cleaning soiled surfaces. Such compositions include but are not limited to, dishwashing compositions, laundry detergent compositions, fabric softening compositions, fabric enhancing compositions, fabric freshening compositions, laundry pre-wash, laundry pretreat, laundry additives, spray products, dry cleaning agent or composition, laundry rinse additive, wash additive, post-rinse fabric treatment, ironing aid, hard surface cleaning compositions, unit dose formulation, delayed delivery formulation, detergent contained on or in a porous substrate or nonwoven sheet, and other suitable forms that may be apparent to one skilled in the art in view of the teachings herein. Such compositions may be used as a pre-cleaning treatment, a post-cleaning treatment, or may be added during the rinse or wash cycle of the cleaning process. The detergent compositions may have a form selected from liquid, powder, single-phase or multi-phase unit dose or pouch form, tablet, gel, paste, bar, or flake. Preferably the composition is for manual washing. The detergent composition of the present invention may be a dishwashing detergent. The composition may be in the form of a liquid. Further non-limiting examples include hard surface cleaners, deodorizers, fabric care compositions, fabric cleaning compositions, manual dish detergents, automatic dish detergents, floor waxes, kitchen cleaners, bathroom cleaners and combinations thereof.

“Cleansing composition” refers to a personal care composition or product intended for use in cleaning a bodily surface such as skin or hair. Some non-limiting examples of cleansing compositions are shampoos, conditioners, conditioning shampoos, shower gels, liquid hand cleansers, facial cleansers, and the like.

A dash (−) preceding or following an atom or capital letter representing a chemical group indicates that the so-designated atom or chemical group has an open valence allowing it to connect to another so-designated atom or chemical group via a covalent bond. For example A-B means that-A-B connects to another atom or group via a covalent bond from A to that other atom or group.

It is understood that the test methods that are disclosed in the Test Methods Section of the present application should be used to determine the respective values of the parameters of Applicants' inventions as described and claimed herein.

It is further understood all listed ranges include any other numerical range, which is narrower and which falls within such broader numerical range, as if such narrower numerical ranges were all expressly written herein.

In all embodiments of the present invention, all percentages are by weight of the total composition, as evident by the context, unless specifically stated otherwise. All ratios are weight ratios, unless specifically stated otherwise, and all measurements are made at 25° C., unless otherwise designated.

### Gluconamide Sulfates

Suitable gluconamide sulfate and sulfonate anionic surfactants have a structure according to formula (I)

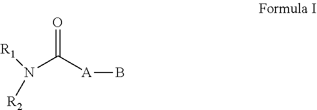

where:
   A=polyhydroxylated alkyl, saccharides;
   B=—OSO₃⁻M⁺, where is M is a cation that forms a metal or organic cation;
   R₁=a heterocyclic, substituted or unsubstituted straight chain or branched chain heteroalkyl, alkyl or alkenyl, having 1 to 22 carbon and/or heteroatoms;
   R₂═H; alkyl;
including all possible stereoisomers thereof:

In some embodiments, R1 has an average alkyl chain length of from about 8 to about 18, from about 10 to about 14, from about 12 to about 14, or from about 12 to about 13 carbon atoms. In some embodiments R₁ is a straight chain alkyl. In embodiments R₂ is a straight chain alkyl that is bio-based, or petroleum-based.

In some embodiments, M is an alkali metal, an alkaline earth metal, ammonium, alkylammonium, or a mixture thereof. In embodiments M is an alkali metal, such as sodium.

The surfactant alcohol precursors can be derived from condensation of amines with hydroxy esters or by ring opening of gluconolactone, which is the dehydration product of glucose, or other sugar lactones and a fatty amine, as described by Arevalo, M. J.; Avalos, M.; Babiano, R.; Cabanillas, A.; Cintas, P.; Jimenez, J. L.; Palacios, J. C; Tetrahedron Asymmetry, 2000, (11), 1985-1995. Such alkyl gluconamide sulfate anionic surfactants can be produced as described in the sulfation chemistry reported by Mehltretter, C. L.; Furry, M. S.; Millies, R. L.; Rankin, J. C.; J. Am. Oil Chemists Soc.; 1952, 202-207.

The alkyl gluconamide sulfate/sulfonate anionic surfactants of Formula I can comprise several different polyhydroxylated groups including sugars. The sugars may include glucose, mannose, galactose, talanose, and allanose.

The gluconamide sulfate anionic surfactant can have a weight average degree of branching of less than 30%, preferably less than 20%, more preferably less than 10%, and most preferably the alkyl chain of the gluconamide sulfate anionic surfactant is linear.

Suitable examples of commercially available alkyl sulfate anionic surfactants include, those derived from alcohols sold under the Neodol® brand-name by Shell Oil Company, The Hague, Netherlands, or the Lial®, Isalchem®, and Safol® brand-names by Sasol, Sandton, South Africa, and Exxal® brand-name by Exxon-Mobil, Irving, Texas, and Lutensol® brand-name by BASF, Ludwigshafen, Germany, or some of the natural alcohols, such as dodecan-1-ol, tetradecan-1-ol, or hexadecan-1-ol produced by The Procter & Gamble Company, Cincinnati, OH.

The surfactant system may comprise further anionic surfactant, including p-alkylbenzene sulfonic acid (HLAS), paraffin sulfonates, olefin sulfonates, methyl ester sulfonates, isethionates, glutamates, sarcosinates, glycinates, taurates, ether carboxylates, sophorolipids, rhamnolipids, or sulfosuccinate anionic surfactants.

The detergent compositions may comprise one or more additional surfactants (e.g., a third surfactant, a fourth surfactant), such as anionic surfactants, nonionic surfactants, cationic surfactants, zwitterionic surfactants, amphoteric surfactants, ampholytic surfactants, and mixtures thereof. The additional surfactant may be a detersive surfactant, which those of ordinary skill in the art will understand to encompass any surfactant or mixture of surfactants that provide sudsing, cleaning, stain removing, or laundering benefit to soiled material.

The gluconamide surfactants may be synthesized in several different ways as has been described previously in the literature and according to the Schemes below.

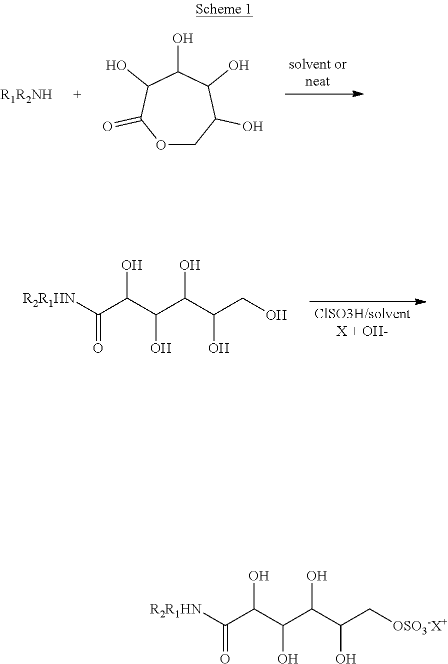

Embodiments of the invention involve the condensation of an appropriate petroleum, such as an amine derived from petro-based olefins and/or acids or naturally derived amine with a lactone derived from dehydration of a natural sugar (Scheme 1), followed by selective sulfation, or the amidation of a hydroxy ester feedstock (Scheme 2), followed by selective sulfation of the terminal hydroxy group.

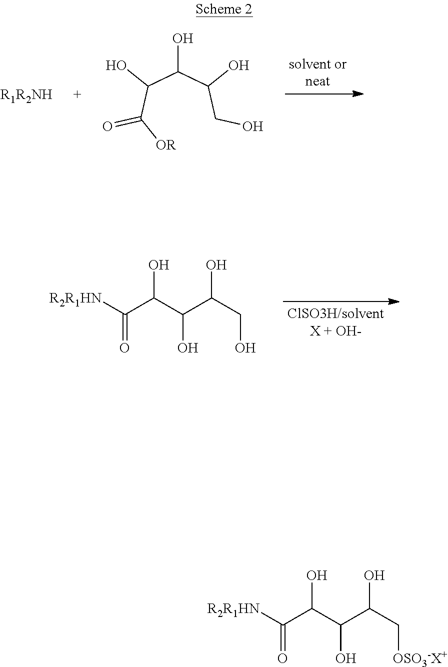

The gluconamide sulfates in TABLE 1 can be synthesized according to the descriptions outlined in either Scheme 1 or Scheme 2.

TABLE 1

| Compound | Name |
|---|---|
| 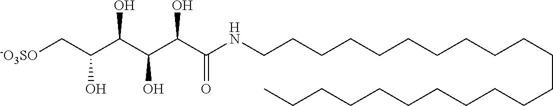 | Sodium (2R,3R,4S,5R)-6-(docosylamino)- 2,3,4,5-tetrahydroxy-6-oxohexyl sulfate |
| 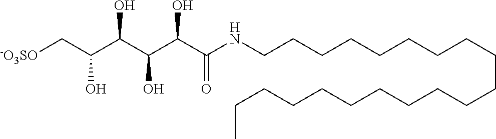 | Sodium (2R,3R,4S,5R)-2,3,4,5-tetrahydroxy-6- (icosylamino)-6-oxohexyl sulfate |
| 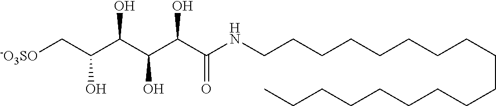 | Sodium (2R,3R,4S,5R)-2,3,4,5-tetrahydroxy-6- (octadecylamino)-6-oxohexyl sulfate |
| 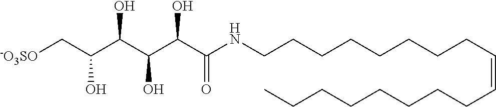 | Sodium (2R,3R,4S,5R)-2,3,4,5-tetrahydroxy-6- (((Z)-octadec-9-en-1-yl) amino)-6-oxohexyl sulfate |
| 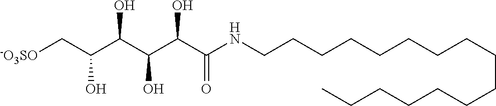 | Sodium (2R,3R,4S,5R)-6-(hexadecylamino)- 2,3,4,5-tetrahydroxy-6-oxohexyl sulfate |
| 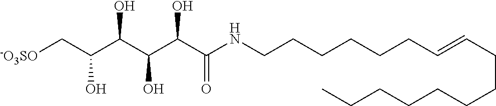 | Sodium (2R,3R,4S,5R)-6-(((E)-hexadec-7-en-1- yl) amino)-2,3,4,5-tetrahydroxy-6-oxohexyl sulfate |
| 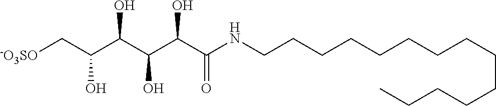 | Sodium (2R,3R,4S,5R)-6-(tetradecylamino)- 2,3,4,5-tetrahydroxy-6-oxohexyl sulfate |
| 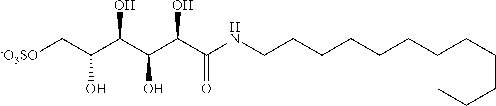 | Sodium (2R,3R,4S,5R)-6-(dodecylamino)- 2,3,4,5-tetrahydroxy-6-oxohexyl sulfate |
| 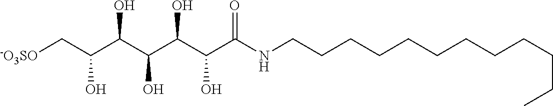 | Sodium (2R,3R,4S,5R)-N-Dodecyl-D-glycero-D- gulo-heptanamide |
| 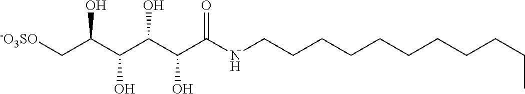 | Sodium (2R,3R,4S,5R)-2,3,4,5-tetrahydroxy-6- (undecylamino)-6-oxohexyl sulfate |
| 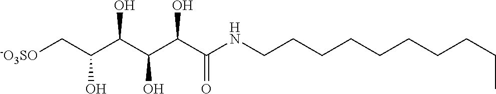 | Sodium (2R,3R,4S,5R)-2,3,4,5-tetrahydroxy-6- (decylamino)-6-oxohexyl sulfate |
| 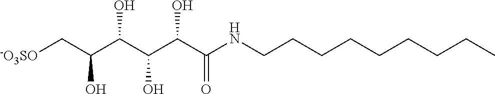 | Sodium (2R,3R,4S,5R)-2,3,4,5-tetrahydroxy-6- (nonylamino)-6-oxohexyl sulfate |
| 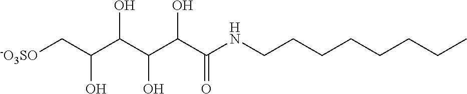 | Sodium (2R,3R,4S,5R)-2,3,4,5-tetrahydroxy-6- (octylamino)-6-oxohexyl sulfate |
| 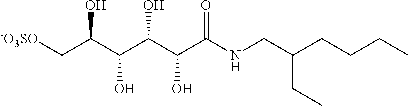 | Sodium (2R,3R,4S,5R)-6-((2-ethylhexyl)amino- 2,3,4,5-tertahydroxy-6-oxohexyl sulfate |
| 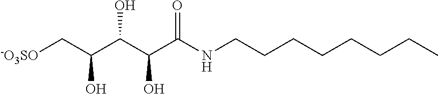 | Sodium (2R,3R,4S,5R)-2,3,4-trihydroxy-5- (octylamino)-5-oxopentyl sulfate |
| 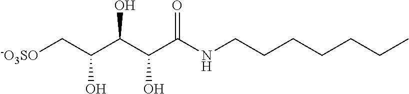 | Sodium (2R,3R,4R)-5-(heptylamino)-2,3,4- trihydroxy-5-oxopentyl sulfate |
| 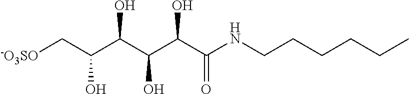 | Sodium (2R,3R,4S,5R)-6-(hexylamino)-2,3,4,5- tetrahydroxy-6-oxohexyl sulfate |
| 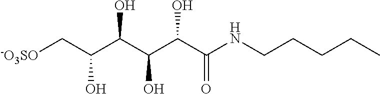 | Sodium (2R,3R,4S,5S)-2,3,4,5-tetrahydroxy-6- oxo-6-(pentylamino)hexyl sulfate |
| 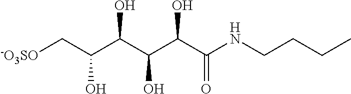 | Sodium (2R,3R,4S,5R)-6-(butylamino)-2,3,4,5- tetrahydroxy-6-oxohexyl sulfate |
| 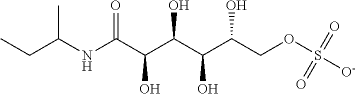 [SMILES: CCC(C)NC(=O)[C@H](O)[C@@H](O)[C@H](O)[C@H](O)COS(=O)(=O)[O-]] | Sodium (2R,3R,4S,5R)-6-(sec-butylamino)- 2,3,4,5-tetrahydroxy-6-oxohexyl sulfate |
| 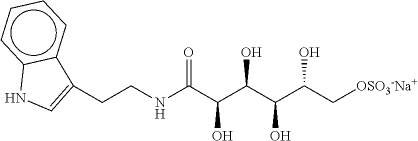 | Sodium (2R,3R,4S,5R)-6-((2-1H-indol-2- yl)ethyl)amino)-2,3,4,5-tetrahydroxy-6-oxohexyl sulfate |
| 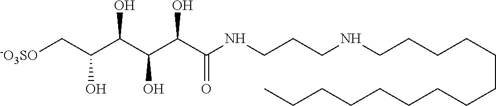 | Sodium (2R,3R,4S,5R)-2,3,4,5-tetrahydroxy-6- oxo-6((3-(tetradecylamino)propyl)amino)hexyl sulfate |
| 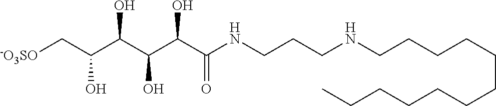 | Sodium (2R,3R,4S,5R)-6-((3- dodecylamino)propyl)amino)-2,3,4,5- tetrahydroxy-6-oxohexyl sulfate |
| 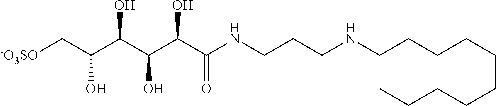 | Sodium (2R,3R,4S,5R)-6-((3- decylamino)propyl)amino)-2,3,4,5-tetrahydroxy- 6-oxohexyl sulfate |
| 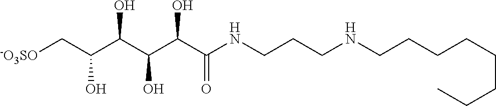 | Sodium (2R,3R,4S,5R)-2,3,4,5-tetrahydroxy-6- ((2-(octylamino)propyl)amino)-6-oxohexyl sulfate |
| 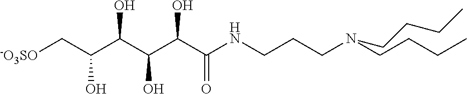 | Sodium (2R,3R,4S,5R)-6-((3- (dibutylamino)propyl)amino)-2,3,4,5- tetrahydroxy-6-oxohexyl sulfate |
| 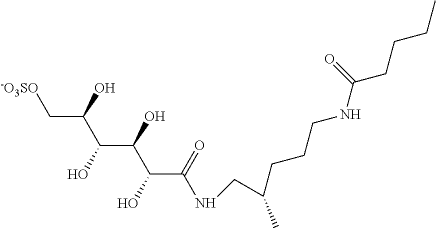 | Sodium (2R,3R,4R,5R)-2,3,4,5-tetrahydroxy-6- (((S)-2-methyl-5-pentanamidopentyl)amino)-6- oxohexyl sulfate |
| 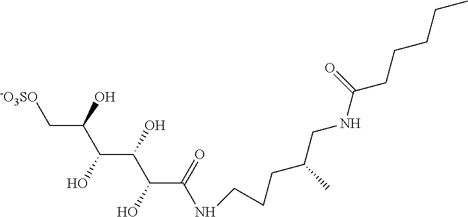 | Sodium (2R,3R,4S,5R)-6-(((R)-5-hexanamido-4- methylpentyl)amino)-2,3,4,5-tetrahydroxy-6- oxohexyl sulfate |
| 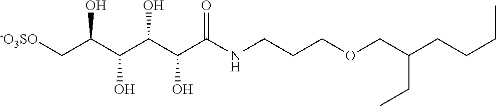 | Sodium (2R,3R,4S,5R)-6-((3-((2- ethylhexyl)oxy)propyl) amino)-2,3,4,5-tetrahydroxy-6-oxohexyl sulfate |
| 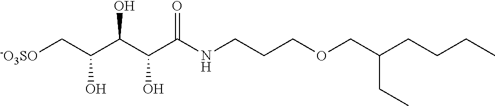 | Sodium (2R,3R,4R)-5-((3-((2- ethylhexyl)oxy)propyl) amino)-2,3,4-trihydroxy-5-oxopentyl sulfate |
| 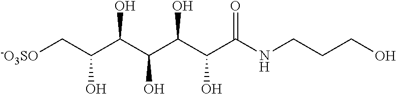 | Sodium (2R,3R,4S,5R,6R)-2,3,4,5,6- pentahydroxy- 7-((3-hydroxypropyl)amino)-7-oxoheptyl sulfate |
| 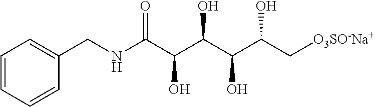 | Sodium (2R,3R,4S,5R)-6-(benzylamino)-2,3,4,5- tetrahydroxy-6-oxohexyl sulfate |

### Liquid Detergent Composition

A detergent composition, which includes a GS of the present invention, may be a hand dishwashing detergent composition in liquid form. The liquid detergent composition is preferably an aqueous detergent composition. As such, the composition can comprise from 50% to 85%, preferably from 50% to 75%, by weight of the total composition of water.

The pH of the composition may be at least 7.0, preferably from about 7.0 to about 12.0, or more preferably from about 7.5 to about 10.0, as measured at 10% dilution in distilled water at 20° C. The pH of the composition can be adjusted using pH modifying ingredients known in the art.

The composition can be Newtonian or non-Newtonian, with certain embodiments being Newtonian. The composition may have a viscosity of from about 10 mPa's to about 10,000 mPa·s, from about 100 mPa·s to about 5,000 mPa·s, from about 300 mPa·s to about 2,000 mPa's, or from about 500 mPa·s to about 1,500 mPa·s, alternatively combinations thereof. The viscosity is measured with a Brookfield RT Viscometer using spindle 21 at 20 RPM at 25° C.

### Surfactant System

A detergent composition can comprise from about 5% to about 50%, from about 8% to about 45%, or from about 15% to about 40%, by weight of the total composition of a surfactant system. The surfactant system comprises anionic surfactant and a co-surfactant which is selected from the group consisting of an amphoteric surfactant, a zwitterionic surfactant and mixtures thereof.

In order to improve surfactant packing after dilution and hence improve robustness against hardness variations and suds mileage, the surfactant system can comprise the anionic surfactant and co-surfactant in a weight ratio of co-surfactant:anionic surfactant from about 1:1 to about 1:9, about 1:2 to about 1:5, or about 1:2.5 to about 1:4.

### Anionic Surfactant:

A detergent composition may comprise from about 60% to about 90%, from about 65% to about 85%, or from about 70% to about 80% by weight of the surfactant system of the anionic surfactant. The anionic surfactant can comprise from about 10% to about 100%, from about 20% to about 80%, or from about 25% to about 35% by weight of the anionic surfactant of a glycerol acetal sulfate or sulfonate anionic surfactant.

### Co-Surfactant:

A detergent composition may further comprise a co-surfactant that is at least one of an amphoteric surfactant, a zwitterionic surfactant or mixtures thereof, as part of the surfactant system. The composition may comprise from about 0.1% to about 20%, from about 0.5% to about 15%, or from about 2% to about 10% by weight of the detergent composition of the co-surfactant.

The surfactant system of the detergent composition may comprise from about 10% to about 40%, from about 15% to about 35%, or from about 20% to about 30%, by weight of the surfactant system of a co-surfactant.

The co-surfactant may be at least one of an amphoteric surfactant, a zwitterionic surfactant, and mixtures thereof. In embodiments the co-surfactant may be an amphoteric surfactant, such as an amine oxide surfactant.

The amine oxide surfactant can be linear or branched, with certain embodiments being linear. Suitable linear amine oxides are typically water-soluble, and characterized by the formula R1—N(R2) (R3) O wherein R1 is a C8-18 alkyl, and the R2 and R3 moieties are selected from the group consisting of C1-3 alkyl groups, C1-3 hydroxyalkyl groups, and mixtures thereof. For instance, R₂ and R₃ can be selected from the group consisting of: methyl, ethyl, propyl, isopropyl, 2-hydroxethyl, 2-hydroxypropyl and 3-hydroxypropyl, and mixtures thereof, wherein in certain embodiments methyl may be one or both of R2 and R3. The linear amine oxide surfactants in particular may include linear C10-C18 alkyl dimethyl amine oxides and linear C10-C18 dihydroxyethyl amine oxides.

The amine oxide surfactant may be at least one of: alkyl dimethyl amine oxide, alkyl amido propyl dimethyl amine oxide, and mixtures thereof. In certain embodiments alkyl dimethyl amine oxides may be used, such as C8-18 alkyl dimethyl amine oxides, or C10-16 alkyl dimethyl amine oxides (such as coco dimethyl amine oxide). Suitable alkyl dimethyl amine oxides include C10 alkyl dimethyl amine oxide surfactant, C10-12 alkyl dimethyl amine oxide surfactant, C12-C14 alkyl dimethyl amine oxide surfactant, and mixtures thereof. In certain embodiments the amine oxide surfactant may be C12-C14 alkyl dimethyl amine oxide and the alkyl chain of the alkyl dimethyl amine oxide may be a linear alkyl chain, such as a C12-C14 alkyl chain, for example a C12-C14 alkyl chain derived from coconut oil, palm kernel oil, or from Ziegler alcohols.

Alternative suitable amine oxide surfactants include mid-branched amine oxide surfactants. As used herein, “mid-branched” means that the amine oxide has one alkyl moiety having n1 (number of C atoms on the branch n1) carbon atoms with one alkyl branch on the alkyl moiety having n2 (number of carbon atoms on the branch n2) carbon atoms. The alkyl branch is located on the α carbon from the nitrogen on the alkyl moiety. This type of branching for the amine oxide is also known in the art as an internal amine oxide. The total sum of n1 and n2 can be from about 10 to about 24 carbon atoms, from about 12 to about 20, or from about 10 to about 16. The number of carbon atoms for the one alkyl moiety (n1) may be the same or similar to the number of carbon atoms as the one alkyl branch (n2) such that the one alkyl moiety and the one alkyl branch are symmetric. As used herein “symmetric” means that |n1−n2| in certain embodiments is less than or equal to about 5, less than or equal to about 4, or from 0 to about 4 carbon atoms in at least 50 wt % or at least 75 wt % to 100 wt % of the mid-branched amine oxides for use herein. The amine oxide further comprises two moieties, independently selected from a C1-3 alkyl, a C1-3 hydroxyalkyl group, or a polyethylene oxide group containing an average of from about 1 to about 3 ethylene oxide groups. The two moieties may be selected from a C1-3 alkyl, more preferably both are selected as C1 alkyl.

In embodiments, the amine oxide surfactant can be a mixture of amine oxides comprising a mixture of low-cut amine oxide and mid-cut amine oxide. The amine oxide can then comprise:
a) from about 10% to about 45% by weight of the amine oxide of low-cut amine oxide of formula R1R2R3AO wherein R1 and R2 are independently selected from hydrogen, C1-C4 alkyls or mixtures thereof, and R3 is selected from C10 alkyls and mixtures thereof; and
b) from about 55% to about 90% by weight of the amine oxide of mid-cut amine oxide of formula R4R5R6AO wherein R4 and R5 are independently selected from hydrogen, C1-C4 alkyls or mixtures thereof, and R6 is selected from C12-C16 alkyls or mixtures thereof.

In embodiments a low-cut amine oxide for use herein R3 is n-decyl, wherein both R1 and R2 may be methyl. In the mid-cut amine oxide of formula R4R5R6AO, R4 and R5 may be both methyl.

In embodiments the amine oxide comprises less than about 5% or less than about 3%, by weight of the amine oxide of an amine oxide of formula R7R8R9AO wherein R7 and R⁸ are selected from hydrogen, C1-C4 alkyls and mixtures thereof and wherein R9 is selected from C8 alkyls and mixtures thereof. Limiting the amount of amine oxides of formula R7R8R9AO improves both physical stability and suds mileage.

Suitable zwitterionic surfactants include betaine surfactants. Such betaine surfactants include alkyl betaines, alkyl N,N-dimethyl betaines, alkylamidobetaine, amidazoliniumbetaine, alkyl sulfobetaines (INCI Sultaines) as well as the alkyl phosphobetaines, and preferably meets formula (IV):

R¹-[CO—X(CH₂)ₙ]ₓ—N⁺(R²)(R₃)—(CH₂)ₘ—[CH(OH)—CH₂]_{y}—Y⁻
wherein in formula (IV),
   R1 is at least one of: a saturated or unsaturated C6-22 alkyl residue, preferably C8-18 alkyl residue, a saturated C10-16 alkyl residue, or a saturated C12-14 alkyl residue;
   X is selected from the group consisting of: NH, NR4 wherein R4 is a C1-4 alkyl residue, O, and S;
   n is an integer from 1 to 10, preferably 2 to 5, more preferably 3′;
   x is 0 or 1;
   R2 and R3 are independently at least one of: a C1-4 alkyl residue, hydroxy substituted such as a hydroxyethyl, and mixtures thereof, preferably both R2 and R3 are methyl;
   m is an integer from 1 to 4;
   y is 0 or 1; and
   Y is at least one of: COO, SO3, OPO(OR5)O or P(O)(OR5)O, wherein R5 is H or a C1-4 alkyl residue.

Betaines may be the alkyl betaines of formula (IVa), the alkyl amido propyl betaine of formula (IVb), the sulfobetaines of formula (IVc) and the amido sulfobetaine of formula (IVd):

R¹—N⁺(CH₃)₂—CH₂COO⁻ (IVa)

R¹—CO—NH—(CH₂)₃—N⁺(CH₃)₂—CH₂COO⁻ (IVb)

R¹—N⁺(CH₃)₂—CH₂CH(OH)CH₂SO₃⁻ (IVc)

R¹—CO—NH—(CH₂)₃—N⁺(CH₃)₂—CH₂CH(OH)CH₂SO₃⁻ (IVd)

in which R1 has the same meaning as in formula (IV). In embodiments the carbobetaines [i.e. wherein Y⁻═COO— in formula (IV)] of formulae (IVa) and (IVb), may be used, such as the alkylamidobetaine of formula (IVb).

Suitable betaines can be selected from the group consisting or [designated in accordance with INCI]: capryl/capramidopropyl betaine, cetyl betaine, cetyl amidopropyl betaine, cocamidoethyl betaine, cocamidopropyl betaine, cocobetaines, decyl betaine, decyl amidopropyl betaine, hydrogenated tallow betaine/amidopropyl betaine, isostearamidopropyl betaine, lauramidopropyl betaine, lauryl betaine, myristyl amidopropyl betaine, myristyl betaine, oleamidopropyl betaine, oleyl betaine, palmamidopropyl betaine, palmitamidopropyl betaine, palm-kernelamidopropyl betaine, stearamidopropyl betaine, stearyl betaine, tallowamidopropyl betaine, tallow betaine, undecylenamidopropyl betaine, undecyl betaine, and mixtures thereof. In embodiments betaines may be at least one of: cocamidopropyl betaine, cocobetaines, lauramidopropyl betaine, lauryl betaine, myristyl amidopropyl betaine, myristyl betaine, and mixtures thereof. In embodiments cocamidopropyl betaine may be used.

### Nonionic Surfactant:

The surfactant system can further comprise a nonionic surfactant. In embodiments a nonionic surfactant may be at least one of: alkyl alkoxylated nonionic surfactants, alkylpolyglucosides, and mixtures thereof. In embodiments the nonionic surfactant may be alkylpolyglucosides.

The surfactant system can comprise from about 1% to about 25%, preferably from about 1.25% to about 20%, more preferably from about 1.5% to about 15%, most preferably from about 1.5% to about 5%, by weight of the surfactant system, of the non-ionic surfactant.

The alkoxylated non-ionic surfactant can be a linear or branched, primary or secondary alkyl alkoxylated non-ionic surfactant, such as an alkyl ethoxylated non-ionic surfactant, which may comprise on average from about 9 to about 15 carbon atoms in its alkyl chain, or from about 10 to about 14 carbon atoms, and on average from about 5 to about 12 units of ethylene oxide per mole of alcohol, from about 6 to about 10 units, or from about 7 to about 8 units.

A detergent composition can comprise alkyl polyglucoside (“APG”) surfactant. The addition of alkyl polyglucoside surfactants have been found to improve sudsing beyond that of comparative nonionic surfactants such as alkyl ethoxylated surfactants.

The alkyl polyglucoside surfactant may be a C8-C16 alkyl polyglucoside surfactant, such as a C8-C14 alkyl polyglucoside surfactant. The alkyl polyglucoside may have an average degree of polymerization of between about 0.1 to about 3, between about 0.5 to about 2.5, or between about 1 to about 2. In embodiments the alkyl polyglucoside surfactant may have an average alkyl carbon chain length between about 10 to about 16, between about 10 to about 14, or between about 12 to about 14, with an average degree of polymerization of between about 0.5 to about 2.5 between about 1 to about 2, or between about 1.2 to about 1.6.

C8-C16 alkyl polyglucosides are commercially available from several suppliers (e.g., Simusol® surfactants from Seppic Corporation, Paris, France; and Glucopon® 600 CSUP, Glucopon® 650 EC, Glucopon® 600 CSUP/MB, and Glucopon® 650 EC/MB, from BASF Corporation, Ludwigshafen, Germany).

### Amphiphilic Alkoxylated Polyalkyleneimine:

A detergent composition may further comprise from about 0.05% to about 2%, preferably from about 0.07% to about 1% by weight of the total composition of an amphiphilic polymer. Suitable amphiphilic polymers can be at least one of: amphiphilic alkoxylated polyalkyleneimine and mixtures thereof. The amphiphilic alkoxylated polyalkyleneimine polymer has been found to reduce gel formation on the hard surfaces to be cleaned when the liquid composition is added directly to a cleaning implement (such as a sponge) before cleaning and consequently brought in contact with heavily greased surfaces, especially when the cleaning implement comprises a low amount to nil water such as when light pre-wetted sponges are used.

The amphiphilic alkoxylated polyalkyleneimine may be an alkoxylated polyethyleneimine polymer comprising a polyethyleneimine backbone having a weight average molecular weight range of from about 100 to about 5,000, from about 400 to about or 2,000, more preferably from about 400 to about 1,000 Daltons. The polyethyleneimine backbone comprises the following modifications:
(i) one or two alkoxylation modifications per nitrogen atom, dependent on whether the modification occurs at an internal nitrogen atom or at an terminal nitrogen atom, in the polyethyleneimine backbone, the alkoxylation modification consisting of the replacement of a hydrogen atom on by a polyalkoxylene chain having an average of about 1 to about 50 alkoxy moieties per modification, wherein the terminal alkoxy moiety of the alkoxylation modification is capped with hydrogen, a C1-C4 alkyl or mixtures thereof;
(ii) a substitution of one C1-C4 alkyl moiety and one or two alkoxylation modifications per nitrogen atom, dependent on whether the substitution occurs at an internal nitrogen atom or at a terminal nitrogen atom, in the polyethyleneimine backbone, the alkoxylation modification consisting of the replacement of a hydrogen atom by a polyalkoxylene chain having an average of about 1 to about 50 alkoxy moieties per modification wherein the terminal alkoxy moiety is capped with hydrogen, a C1-C4 alkyl or mixtures thereof; or
(iii) a combination thereof.

For example, but not limited to, shown below are possible modifications to terminal nitrogen atoms in the polyethyleneimine backbone where R represents an ethylene spacer and E represents a C1-C4 alkyl moiety and X-represents a suitable water soluble counterion:

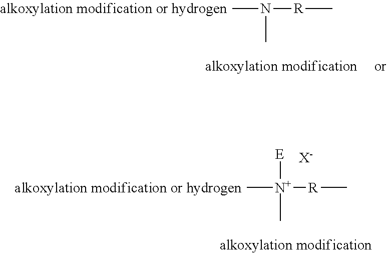

Also, for example, but not limited to, shown below are possible modifications to internal nitrogen atoms in the polyethyleneimine backbone where R represents an ethylene spacer and E represents a C1-C4 alkyl moiety and X-represents a suitable water soluble counterion:

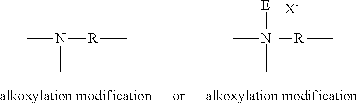

The alkoxylation modification of the polyethyleneimine backbone consists of the replacement of a hydrogen atom by a polyalkoxylene chain having an average of about 1 to about 50 alkoxy moieties, from about 20 to about 45 alkoxy moieties, or from about 30 to about 45 alkoxy moieties. The alkoxy moieties may be from ethoxy (EO), propoxy (PO), butoxy (BO), and mixtures thereof. The polyalkoxylene chain may be selected from ethoxy/propoxy block moieties. In embodiments, the polyalkoxylene chain is ethoxy/propoxy block moieties having an average degree of ethoxylation from about 3 to about 30 and an average degree of propoxylation from about 1 to about 20, or ethoxy/propoxy block moieties having an average degree of ethoxylation from about 20 to about 30 and an average degree of propoxylation from about 10 to about 20.

In embodiments the ethoxy/propoxy block moieties have a relative ethoxy to propoxy unit ratio between about 3 to about 1 and about 1 to 1, in certain embodiments between about 2 to about 1 and about 1 to about 1.

The modification may result in permanent quaternization of the polyethyleneimine backbone nitrogen atoms. The degree of permanent quaternization may be from 0% to about 30% of the polyethyleneimine backbone nitrogen atoms. In embodiments there may be less than 30% of the polyethyleneimine backbone nitrogen atoms permanently quaternized. In embodiments the degree of quaternization can be close to 0% or 0%.

An amphiphilic alkoxylated polyethyleneimine polymer may have the general structure of formula (II):

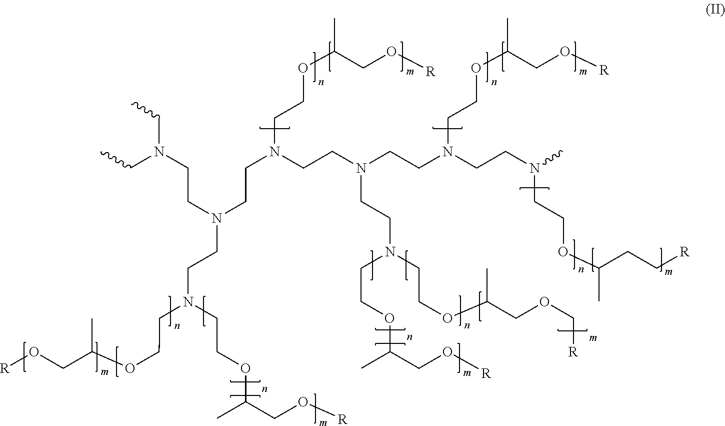

wherein the polyethyleneimine backbone has a weight average molecular weight of about 600, n of formula (II) has an average of about 10 meaning the oligomeric blocks of ethylene oxide have about 10 units of ethylene oxide on average m of formula (II) has an average of about 7 meaning the oligomeric blocks of propylene oxide have about 7 units of propylene oxide on average, and R of formula (II) is at least one of hydrogen, a C1-C4 alkyl and mixtures thereof, in certain embodiments hydrogen. The degree of permanent quaternization of formula (II) may be from 0% to about 22% of the polyethyleneimine backbone nitrogen atoms. The molecular weight of this amphiphilic alkoxylated polyethyleneimine polymer preferably is between 10,000 and 15,000 Da.

In embodiments, the amphiphilic alkoxylated polyethyleneimine polymer has the general structure of formula (II) but wherein the polyethyleneimine backbone has a weight average molecular weight of about 600 Da, n of Formula (II) has an average of about 24 meaning the oligomeric blocks of ethylene oxide have about 24 units of ethylene oxide on average, m of Formula (II) has an average of about 16 meaning the oligomeric blocks of propylene oxide have about 16 units of propylene oxide on average, and R of Formula (II) is at least one of hydrogen, a C1-C4 alkyl and mixtures thereof, in certain embodiments hydrogen. The degree of permanent quaternization of Formula (II) may be from 0% to about 22% of the polyethyleneimine backbone nitrogen atoms, in embodiments it may be 0%. The molecular weight of this amphiphilic alkoxylated polyethyleneimine polymer may be between about 25,000 to about 30,000, in embodiments about 28,000 Da.

The amphiphilic alkoxylated polyethyleneimine polymers can be made by the methods described in more detail in PCT Publication No. WO 2007/135645.

### Cyclic Polyamine

A detergent composition can comprise a cyclic polyamine having amine functionalities that helps cleaning. The composition may comprise from about 0.1% to about 3%, from about 0.2% to about 2%, or from about 0.5% to about 1%, by weight of the composition, of the cyclic polyamine.

The amine can be subjected to protonation depending on the pH of the cleaning medium in which it is used. Preferred cyclic polyamines have the following Formula (III):

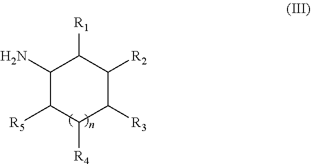

wherein R₁, R₂, R₃, R₄ and R₅ are independently at least one of NH2, —H, linear or branched alkyl having from about 1 to about 10 carbon atoms, and linear or branched alkenyl having from about 1 to about 10 carbon atoms, n is from about 1 to about 3, and wherein at least one of the Rs is NH2 and the remaining “Rs” are independently selected from the group consisting of NH2, —H, linear or branched alkyl having about 1 to about 10 carbon atoms, and linear or branched alkenyl having from about 1 to about 10 carbon atoms. The cyclic polyamine may be a diamine, wherein n is 1, R₂ is NH2, and at least one of R₁, R₃, R₄ and R₅ is CH₃ and the remaining Rs are H.

The cyclic polyamine has at least two primary amine functionalities. The primary amines can be in any position in the cyclic amine but it has been found that in terms of grease cleaning, better performance is obtained when the primary amines are in positions 1,3. It has also been found that cyclic amines in which one of the substituents is —CH₃ and the rest are H provided for improved grease cleaning performance.

Accordingly, in embodiments the cyclic polyamine for use with a detergent are cyclic polyamine may be at least one of: 2-methylcyclohexane-1,3-diamine, 4-methylcyclohexane-1,3-diamine and mixtures thereof. These specific cyclic polyamines work to improve suds and grease cleaning profile through-out the dishwashing process when formulated together with the surfactant system of the composition of the present invention.

### Additional Ingredients:

A detergent composition may further comprise at least one active comprising: i) a salt, ii) a hydrotrope, iii) an organic solvent, or mixtures thereof.

### Salt:

The composition may comprise from about 0.05% to about 2%, from about 0.1% to about 1.5%, or from about 0.5% to about 1%, by weight of the total composition of a salt, in embodiments a monovalent or divalent inorganic salt, or a mixture thereof, for example sodium chloride, sodium sulfate, or mixtures thereof.

### Hydrotrope:

The composition may comprise from about 0.1% to about 10%, from about 0.5% to about 10%, or from about 1% to about 10% by weight of the total composition of a hydrotrope or a mixture thereof, for example sodium cumene sulfonate.

### Organic Solvent:

A composition can comprise from about 0.1% to about 10%, from about 0.5% to about 10%, or from about 1% to about 10% by weight of the total composition of an organic solvent. Suitable organic solvents include: alcohols, glycols, glycol ethers, and mixtures thereof, preferably alcohols, glycols, and mixtures thereof. In embodiments ethanol is the organic solvent. In embodiments polyalkyleneglycols, such as polypropyleneglycol, are the organic solvents.

### Adjunct Ingredients

A detergent composition may optionally comprise a number of other adjunct ingredients such as builders (preferably citrate), chelants, conditioning polymers, other cleaning polymers, surface modifying polymers, structurants, emollients, humectants, skin rejuvenating actives, enzymes, carboxylic acids, scrubbing particles, perfumes, perfume delivery aids, enzymes, hueing dyes, malodor control agents, pigments, dyes, opacifiers, pearlescent particles, inorganic cations such as alkaline earth metals such as Ca/Mg-ions, antibacterial agents, preservatives, viscosity adjusters (e.g., salt such as NaCl, and other mono-, di- and trivalent salts) and pH adjusters and buffering means (e.g. carboxylic acids such as citric acid, HCl, NaOH, KOH, alkanolamines, carbonates such as sodium carbonates, bicarbonates, sesquicarbonates, and analogs).

### Test Methods

The following assays set forth are used in order that the invention described and claimed herein may be more fully understood.

### Suds Generation and Suds Mileage Test Method

The suds generation and suds mileage of test cleaning compositions herein is measured by employing a suds cylinder tester (SCT). The SCT has a set of 8 cylinders. Each cylinder is a Lexan plastic cylinder that is 30 cm long and 8.8 cm internal diameter with an adhesive ruler affixed to the outside, and a small diameter hole in the top to enable soil additions. Cylinders are together rotated at a rate of 20-22 revolutions per minute (rpm). This method is used to assay the performance of test cleaning compositions to obtain a reading on ability to generate suds as well as the robustness of the suds in the presence of test soil. Approximately 500 ml of the test cleaning solutions are prepared at a surfactant concentration of 359 mg/L in water heated to 60° C. and a water hardness of about 257 mg/L made using calcium chloride and magnesium chloride at a 3:1 molar ratio of calcium:magnesium. 300 ml of each test sample solution is poured into a test cylinder in the SCT. When the test solutions have cooled to 45° C., rubber stoppers are put in place to seal the hole in the top of each cylinder.

Rotate cylinders for 2 minutes. Lock in an upright position Record initial suds height for each cylinder. The height of suds is determined by deducting the height of the liquid layer from the total height of suds and liquid. Continue rotating the cylinders, recording suds height every 2 minutes for a total of 20 minutes. This data represents the Suds Generation of the test cleaning composition. Open the rubber stopper on each cylinder. Add 10.00 g of test soil into each cylinder. Replace the rubber stoppers. Record the starting suds height, and rotate cylinders for 1 minute. Lock in an upright position. Record suds height. Continue rotating the cylinders, recording suds height every 1 minute for a total of 15 minutes. This data represents the Suds Mileage of the test cleaning composition.

Data is recorded as suds generation or suds mileage (cm) vs time (min). Area under the curve (AUC) is calculated using suds generation or suds mileage vs time data and using the trapezoidal rule calculation:

$$\text{?} \frac{\text{?}}{\text{?}} \text{?}$$

$$\text{?} \text{indicates text missing or illegible when filed}$$

The AUC results for Suds Generation and Suds Mileage for each test solution are divided by the corresponding AUC result for the relevant test reference and reported as an index (%) compared to the control (100%).

Preparation of the test soil is achieved by standard mixing of the components described below until a homogenous mixture is achieved.

TABLE 2

| Ingredient | Weight % |
|---|---|
| Crisco Oil | 12.730 |
| Crisco shortening | 27.752 |
| Lard | 7.638 |
| Refined Rendered Edible | 51.684 |
| Beef Tallow | |
| Oleic Acid, 90% (Techn) | 0.139 |
| Palmitic Acid, 99+% | 0.036 |
| Stearic Acid, 99+% | 0.021 |

### Dynamic Interfacial Tension (DIFT) Test Method

Dynamic Interfacial Tension analysis is performed on a Krüss® DVT30 Drop Volume Tensiometer (Krüss USA, Charlotte, NC). The instrument is configured to measure the interfacial tension of an ascending oil drop in aqueous surfactant (surfactant) phase. The test surfactant solutions are prepared at a surfactant concentration of 359 mg/L in water and a water hardness of about 120 mg/L made using calcium chloride and magnesium chloride at a 3:1 molar ratio of calcium:magnesium. The oil used is canola oil (Crisco Pure Canola Oil manufactured by The J. M. Smucker Company). The aqueous surfactant and oil phases are temperature controlled at 22° C. (+/−1° C.), via a recirculating water temperature controller attached to the tensiometer. A dynamic interfacial tension curve is generated by dispensing the oil drops into the aqueous surfactant phase from an ascending capillary with an internal diameter of 0.2540 mm, over a range of flow rates and measuring the interfacial tension at each flow rate. Data is generated at oil dispensing flow rates of 500 uL/min to 1 uL/min with 2 flow rates per decade on a logarithmic scale. Interfacial tension is measured on three oil drops per flow rate and then averaged. Interfacial tension is reported in units of mN/m. Surface age of the oil drops at each flow rate is also recorded and plots may be generated either of interfacial tension (y-axis) versus oil flow rate (x-axis) or interfacial tension (y-axis) versus oil drop surface age (x-axis). Minimum interfacial tension (mN/m) is the lowest interfacial tension at the slowest flow rate, with lower numbers indicating improved performance. Based on instrument reproducibility, differences greater than 0.1 mN/m are significant for interfacial tension values of less than 1 mM/m.

### Minimum Surface Tension and Critical Micelle Concentration

Surface tension is measured using the Kibron Delta-8 DyneProbe (Kibron Inc., Helsinki, Finland). Before every run, the DyneProbes are heated using the Kibron DyneClean furnace (Kibron Inc., Helsinki, Finland). The bottom end of each probe is brought into contact onto a (very) hot surface. Upon contact, the tip of the probe is heated to around 600° C. This ensures consistent and repeatably clean surfaces.

The critical micelle concentration (CMC) of the surfactant is defined by plotting the surface tension of respective surfactant solutions as a function of the logarithm of surfactant concentration at the desired water temperature (21° C.) and hardness of about 120 mg/L made using calcium chloride and magnesium chloride at a 3:1 molar ratio of calcium:magnesium. The point where the surface versus surfactant concentration slope changes is defined as the CMC value.

### A Typical CMC Determination Comprises:

1: Dispensing aliquots of solution: Liquid handling is done using a pipetting robot.
2: Probe cleaning: The DyneProbes are heated to red hot to burn off all contaminants.
3: Immersion and withdrawal: The DyneProbes are positioned above a row of 8 wells and immersed gently. The force on the plate is measured continuously (400 times/s) and upon withdrawal the surface tension is determined.
4: Analysis: Up to 96 data points are acquired in around 3 minutes. These are automatically logged to a PC and the CMC is determined.

The biodegradability of the salt of the gluconamide sulfate can be tested according to the following method. The Biodegradability Test Method is based on the Organization for Economic Co-operation and Development (OECD) 301B CO₂ evolution (modified Sturm Test) biodegradation test method that screens chemicals for ready biodegradability in anaerobic aqueous medium. In this test, the test substance is suspended in a phosphate buffered nutrient salts media containing an activated sludge inoculum and the formation of carbon dioxide is measured via a closed-circuit respirometer. The test substance is the sole carbon and energy source and under aerobic conditions microorganisms metabolize organic substances producing CO₂ as the ultimate product. The test system is dosed with a known quantity of carbon and biodegradation is calculated as the percent of theoretical CO₂ formation. The test typically runs for 28 days. Ready biodegradability is a regulator definition that is used to classify materials that meet the pass criteria as described in the OECD 301B guideline. Specifically, the following pass level of biodegradation, obtained within 28 days, may be regarded as evidence of ready biodegradability: 60% theoretical carbon dioxide (ThCO₂). The pass level has to be reached in a 10-day window within the 28-day period of the test. The 10-day window begins when the degree of biodegradation 25 has reached 10% ThCO₂ and ends 10 days later or at day 28 of the test, whichever comes first.

TABLE 3
(Biodegradability: Sodium (2R,3R,4S,5R)-6-(dodecylamino)-
2,3,4,5-tetrahydroxy-6-oxohexyl sulfate vs
Glucotain ® Sulfate)

| | % SOC | % THCO₂ |
|---|---|---|
| | Average | average |
| N-Alkanoyl N-methyl glucamide sulfate | 56% | ?? |
| C-12 (straight) Glucotain ® Sulfate | | |
| Sodium (2R,3R,4S,5R)-6-(dodecylamino)- | 8% | 79.6% |
| 2,3,4,5-tetrahydroxy-6-oxohexyl sulfate | | |

In the TABLE 3 above, significant differences in biodegradability were seen, based on structural changes. Sodium (2R,3R,4S,5R)-6-(dodecylamino)-2,3,4,5-tetrahydroxy-6-oxohexyl sulfate provided superior biodegradability than to Glucotain sulfate in this regard.

TABLE 4

| | COMPARATIVE | INVENTIVE | INVENTIVE |
|---|---|---|---|
| | COMPOSITION A1 | COMPOSITION A2 | COMPOSITION A3 |
| | % wt. | % wt. | % wt. |
| RAW MATERIAL | Active | Active | Active |
| Alkyl Sulfate | 16.48 | 16.48 | 16.49 |
| (sodium salt) | | | |
| Alkyl Ethoxy | 7.06 | 0 | 0 |
| Sulfate | | | |
| (sodium salt) | | | |
| Lauryl Dimethyl | 6.36 | 6.36 | 6.36 |
| Amine Oxide | | | |
| Dodecyl | 0 | 7.06 | 0 |
| gluconamide, | | | |
| 6-sodium | | | |
| sulfate | | | |
| Tetradecyl | 0 | 0 | 7.05 |
| gluconamide, | | | |
| 6-sodium | | | |
| sulfate | | | |
| Ethanol | 2.5 | 3.11 | 3.6 |
| NaCl | 0.7 | 0.7 | 0.7 |
| HOD-base | 0.276 | 0.5 | 0.4 |
| Polypropylene | 1.05 | 0.9 | 1.2 |
| glycol | | | |
| MW = 2000 | | | |
| Phenoxyethanol | 0.15 | 0.08 | 0.08 |
| Acticide | 0.0075 | 0.0035 | 0.0075 |
| Dye | 0.00163 | 0.001625 | 0.001625 |
| Perfume | 0 | 0.4 | 0.2 |
| water | to balance | to balance | to balance |

Grease cleaning in a hand dish context was measured to determine optimal chain length for performance for detergent compositions A2 and A3 and compared with the performance of detergent composition A1, as described in TABLE 4.

TABLE 5

| Surfactant ppm | 1496 | 2991 | 4487 | 8973 | 14955 | 29910 |
|---|---|---|---|---|---|---|
| product dilution % | 0.5 | 1 | 1.5 | 3 | 5 | 10 |
| COMPARATIVE | 68.4 | 82.9 | 85.7 | 58.2 | 55.5 | 56.5 |
| COMPOSITION A1 | | | | | | |
| INVENTIVE | 36.1 | 57.1 | 91.9 | 91.2 | 89.3 | 85.6 |
| COMPOSITION A2 | | | | | | |
| INVENTIVE | 47.6 | 46.8 | 61.1 | 72.3 | 79.8 | 78.3 |
| COMPOSITION A3 | | | | | | |

In TABLE 5 above, a significant improvement in grease cleaning benefit with C12 and C14 gluconamide sulfate (C12 and C14 GS) can be identified when compared to the reference sample that contains AES.

### Technical Stain Removal Wash Procedure

The method involves the use of a tergotometer to simulate the washing of fabrics in a washing machine. Test formulations were used to wash the technical stained swatches (9 grams) together with soil SBL fabric (4 grams) and clean knitted cotton ballast (47 grams). Technical stain swatches of CW120 cotton containing stains were purchased from Advanced Product Design Co., Inc (Cincinnati, OH). The wash tests consisted of two internal and four external replicates for each stain type and treatment.

Detergent A-J (Comparative Compositions A, B, F, G and Inventive Compositions C, D, E, H, I, J-TABLE 6) are added to tergotometer pots containing 1 L of the test wash solution plus test fabrics, soiled fabric and clean fabric ballast at 15° C. and 8 US grains per gallon of hardness to deliver 200 mg/L of surfactant (A-E compositions) and 400 mg/L of surfactant (F-J compositions). The tergitometer pots were agitated at 208 rpm for 17 minutes and spun dry. Fabrics were then rinsed in 15° C. water at 8 US grains per gallon at 208 rpm for 5 minutes and spun dry. After the rinse, fabrics were machine dried on timed-dry for 40 minutes in a Whirlpool LER3622PQ2 dryer before being analyzed.

TABLE 6

| | Comparative | Comparative | Inventive | Inventive | Inventive |
|---|---|---|---|---|---|
| | Composition | Composition | Composition | Composition | Composition |
| | A | B | C | D | E |
| | % wt. | % wt. | % wt. | % wt. | % wt. |
| Raw Material | Active | Active | Active | Active | Active |
| Sodium Lauryl | 17 | 0 | 0 | 0 | 0 |
| Sulfate (SLS) | | | | | |
| Sodium Neodol 12- | 0 | 17 | 0 | 0 | 0 |
| 15 Ethoxy (2.5) | | | | | |
| Sulfate (aka | | | | | |
| Sodium Neodol 25 | | | | | |
| AE2.5RS) | | | | | |
| Dodecyl | 0 | 0 | 17 | 0 | 0 |
| gluconamide, 6- | | | | | |
| sodium sulfate | | | | | |
| Tetradecyl | 0 | 0 | 0 | 17 | 0 |
| gluconamide, 6- | | | | | |
| sodium sulfate | | | | | |
| Hexadecyl | 0 | 0 | 0 | 0 | 17 |
| gluconamide, 6- | | | | | |
| sodium sulfate | | | | | |
| Amylase 1 | 0.0225 | 0.0225 | 0.0225 | 0.0225 | 0.0225 |
| Protease2 | 0.0738 | 0.0738 | 0.0738 | 0.0738 | 0.0738 |
| Mannanase3 | 0.0048 | 0.0048 | 0.0048 | 0.0048 | 0.0048 |
| Pectate Lyase4 | 0.0032 | 0.0032 | 0.0032 | 0.0032 | 0.0032 |
| Ethoxylated | 1.28 | 1.28 | 1.28 | 1.28 | 1.28 |
| polyethyleneimine5 | | | | | |
| Ethoxylated- | 1.28 | 1.28 | 1.28 | 1.28 | 1.28 |
| Propoxylated | | | | | |
| polyethyleneimine | | | | | |
| Tetrasodium | 0.60 | 0.60 | 0.60 | 0.60 | 0.60 |
| glutamate | | | | | |
| diacetate | | | | | |
| Mono Ethanol | 2.55 | 2.55 | 2.55 | 2.55 | 2.55 |
| Amine | | | | | |
| Water, Solvents, | Balance | Balance | Balance | Balance | Balance |
| Caustic & Minors | to 100% | to 100% | to 100% | to 100% | to 100% |
| (dye, preservatives, | | | | | |
| Fragrance etc.) | | | | | |

| | Comparative | Comparative | Inventive | Inventive | Inventive |
|---|---|---|---|---|---|
| | Composition | Composition | Composition | Composition | Composition |
| | F | G | H | I | J |
| | % wt. | % wt. | % wt. | % wt. | % wt. |
| Raw Material | Active | Active | Active | Active | Active |
| Sodium Lauryl | 34 | 0 | 0 | 0 | 0 |
| Sulfate (SLS) | | | | | |
| Sodium Neodol 12-15 | 0 | 34 | 0 | 0 | 0 |
| Ethoxy (2.5) Sulfate | | | | | |
| (aka Sodium Neodol | | | | | |
| 25 AE2.5RS) | | | | | |
| Dodecyl gluconamide, | 0 | 0 | 34 | 0 | 0 |
| 6-sodium sulfate | | | | | |
| Tetradecyl | 0 | 0 | 0 | 34 | 0 |
| gluconamide, 6- | | | | | |
| sodium sulfate | | | | | |
| Hexadecyl | 0 | 0 | 0 | 0 | 34 |
| gluconamide, 6- | | | | | |
| sodium sulfate | | | | | |
| Amylase ¹ | 0.0225 | 0.0225 | 0.0225 | 0.0225 | 0.0225 |
| Protease² | 0.0738 | 0.0738 | 0.0738 | 0.0738 | 0.0738 |
| Mannanase³ | 0.0048 | 0.0048 | 0.0048 | 0.0048 | 0.0048 |
| Pectate Lyase⁴ | 0.0032 | 0.0032 | 0.0032 | 0.0032 | 0.0032 |
| Ethoxylated | 1.28 | 1.28 | 1.28 | 1.28 | 1.28 |
| polyethyleneimine⁵ | | | | | |
| Ethoxylated- | 1.28 | 1.28 | 1.28 | 1.28 | 1.28 |
| Propoxylated | | | | | |
| polyethyleneimine | | | | | |
| Tetrasodium | 0.60 | 0.60 | 0.60 | 0.60 | 0.60 |
| glutamate | | | | | |
| diacetate | | | | | |
| Mono Ethanol Amine | 2.55 | 2.55 | 2.55 | 2.55 | 2.55 |
| Water, Solvents, | Balance | Balance | Balance | Balance | Balance |
| Caustic & Minors | to 100% | to 100% | to 100% | to 100% | to 100% |
| (dye, preservatives, | | | | | |
| Fragrance etc.) | | | | | |

¹Amylase enzyme is supplied by Novozymes
²Protease available from DuPont-Genencor, Palo Alto, CA.
³Mannanase is available from Novozymes, Copenhagen, Denmark
⁴Pectate Lyase is available from Novozymes, Copenhagen, Denmark
⁵PE-20 commercially available from BASF

In TABLES 7 and 8 image analysis was used to compare each stain to an unstained fabric control. Software converted images taken into standard colorimetric values and compared these to standards based on the commonly used Macbeth Color Rendition Chart, assigning each stain a colorimetric value (Stain Level). Two internal replicates and four external replicates of each were prepared.

Stain removal from the swatches was measured as follows:

Stain Removal Index(SRI)=(ΔEinitial−ΔEwashed)×100/ΔEinitial

Statistical significance of the differences in stain removal were assessed at 95% confidence.

TABLE 7
Laundry data for GS (@200 ppm), Hydrophobe = C12, 14, 16

| | | | Sodium | | | Dodecyl | | | Tetradecyl | | | Hexadecyl | | |
|---|---|---|---|---|---|---|---|---|---|---|---|---|---|---|
| SRI's @ | | | Neodol 25 | | | gluconamide, 6- | | | gluconamide, 6- | | | gluconamide, 6- | | |
| 200 ppm | SLS | | AE2.5RS | | | sodium sulfate | | | sodium sulfate | | | sodium sulfate | | |
| Stain | A | ASig | B | DelB | BSig | C | DelC | CSig | D | DelD | DSig | E | DelE | ESig |
| APD | 44.9 | | 50.9 | 6.0 | AJ | 49.6 | 4.8 | A | 48.6 | 3.7 | | 49.8 | 4.9 | A |
| Bacon | | | | | | | | | | | | | | |
| Grease | | | | | | | | | | | | | | |
| Dyed | | | | | | | | | | | | | | |
| CW120 | | | | | | | | | | | | | | |
| GSRTBGD001 | | | | | | | | | | | | | | |
| APD | 61.2 | | 72.9 | 11.7 | AC | 63.5 | 2.4 | | 73.5 | 12.4 | AC | 72.5 | 11.4 | AC |
| Black | | | | | | | | | | | | | | |
| Todd Clay | | | | | | | | | | | | | | |
| CW120 | | | | | | | | | | | | | | |
| GSRTBT001 | | | | | | | | | | | | | | |
| APD | 25.3 | | 50.6 | 25.3 | ACDEFHJ | 29.4 | 4.1 | | 38.1 | 12.8 | AC | 36.1 | 10.8 | AC |
| Burnt | | | | | | | | | | | | | | |
| Butter | | | | | | | | | | | | | | |
| CW120 | | | | | | | | | | | | | | |
| GSRTBB001 | | | | | | | | | | | | | | |
| APD | 27.7 | | 40.9 | 13.2 | ACF | 28.5 | 0.8 | | 38.5 | 10.8 | ACF | 39.9 | 12.2 | ACF |
| Cooked | | | | | | | | | | | | | | |
| Beef | | | | | | | | | | | | | | |
| CW120 | | | | | | | | | | | | | | |
| GSRTCB001 | | | | | | | | | | | | | | |
| APD | 50.3 | | 65.3 | 15.0 | ACDEFHJ | 50.8 | 0.4 | | 52.7 | 2.4 | | 56.9 | 6.6 | ACF |
| Grass | | | | | | | | | | | | | | |
| CW120 | | | | | | | | | | | | | | |
| GSRTGR001 | | | | | | | | | | | | | | |
| APD | 30.9 | | 47.0 | 16.1 | ACDH | 29.7 | −1.2 | | 33.4 | 2.5 | | 36.0 | 5.1 | |
| Makeup | | | | | | | | | | | | | | |
| Covergirl | | | | | | | | | | | | | | |
| CW120 | | | | | | | | | | | | | | |
| GSRTCGM001 | | | | | | | | | | | | | | |
| APD Red | 30.0 | | 33.0 | 2.9 | A | 31.4 | 1.4 | | 33.4 | 3.4 | A | 34.0 | 4.0 | AC |
| Wine Wolf | | | | | | | | | | | | | | |
| Blas | | | | | | | | | | | | | | |
| CW120 | | | | | | | | | | | | | | |
| GSRTRW001 | | | | | | | | | | | | | | |
| APD | 70.2 | | 73.6 | 3.4 | | 71.2 | 1.0 | | 72.1 | 1.9 | | 72.1 | 2.0 | |
| Spaghetti | | | | | | | | | | | | | | |
| Sauce | | | | | | | | | | | | | | |
| Ragu | | | | | | | | | | | | | | |
| CW120 | | | | | | | | | | | | | | |
| GSRTSS001 | | | | | | | | | | | | | | |
| APD Tea | 8.3 | | 11.5 | 3.2 | | 9.9 | 1.5 | | 11.2 | 2.9 | | 12.2 | 3.9 | |
| American | | | | | | | | | | | | | | |
| Lipton | | | | | | | | | | | | | | |
| CW120 | | | | | | | | | | | | | | |
| GSRTLIT001 | | | | | | | | | | | | | | |
| CFT | 1.5 | C | 31.5 | 30.0 | ACH | −25.0 | −26.5 | | 25.6 | 24.1 | ACH | 28.2 | 26.7 | ACH |
| ASTM | | | | | | | | | | | | | | |
| Dust | | | | | | | | | | | | | | |
| Sebum | | | | | | | | | | | | | | |
| PCS94 | | | | | | | | | | | | | | |
| CFT Choc | 22.7 | | 28.2 | 5.5 | AC | 22.4 | −0.3 | | 26.9 | 4.3 | | 27.4 | 4.7 | C |
| Soy Milk | | | | | | | | | | | | | | |
| CS45 | | | | | | | | | | | | | | |
| CFT | 18.5 | C | 39.6 | 21.2 | ACDEFHIJ | −4.0 | 22.4 | | 28.0 | 9.5 | ACH | 28.3 | 9.8 | ACH |
| Discriminating | | | | | | | | | | | | | | |
| Sebum | | | | | | | | | | | | | | |
| PCS132 | | | | | | | | | | | | | | |
| CFT Rice | 70.3 | | 72.5 | 2.2 | | 71.0 | 0.7 | | 73.2 | 2.9 | | 70.5 | 0.2 | |
| Starch | | | | | | | | | | | | | | |
| CS28 | | | | | | | | | | | | | | |

TABLE 8
Laundry data for GS (@400 ppm) Hydrophobe = C12, 14, 16

| | | | Sodium | | Dodecyl | | Tetradecyl | | Hexadecyl | |
|---|---|---|---|---|---|---|---|---|---|---|
| SRI's @ | | | Neodol 25 | | gluconamide, 6- | | gluconamide, 6- | | gluconamide, 6- | |
| 400 ppm | SLS | | AE2.5RS | | sodium sulfate | | sodium sulfate | | sodium sulfate | |
| APD | F | DelF | G | DelG | H | DelH | I | DelI | J | DelJ |
| Bacon | | | | | | | | | | |
| Grease | | | | | | | | | | |
| Dyed | | | | | | | | | | |
| CW120 | | | | | | | | | | |
| GSRTBGD001 | | | | | | | | | | |
| APD | 48.3 | 3.4 | 53.7 | 8.8 | 49.6 | 4.8 | 49.5 | 4.6 | 45.8 | 0.9 |
| Black | | | | | | | | | | |
| Todd | | | | | | | | | | |
| Clay | | | | | | | | | | |
| CW120 | | | | | | | | | | |
| GSRTBT001 | | | | | | | | | | |
| APD | 69.6 | 8.4 | 75.4 | 14.3 | 69.0 | 7.8 | 72.1 | 10.9 | 72.3 | 11.2 |
| Burnt | | | | | | | | | | |
| Butter | | | | | | | | | | |
| CW120 | | | | | | | | | | |
| GSRTBB001 | | | | | | | | | | |
| APD | 34.9 | 9.6 | 64.3 | 39.0 | 32.4 | 7.1 | 52.5 | 27.2 | 37.3 | 12.1 |
| Cooked | | | | | | | | | | |
| Beef | | | | | | | | | | |
| CW120 | | | | | | | | | | |
| GSRTCB001 | | | | | | | | | | |
| APD | 30.5 | 2.9 | 63.1 | 35.4 | 38.9 | 11.3 | 43.2 | 15.5 | 42.1 | 14.5 |
| Grass | | | | | | | | | | |
| CW120 | | | | | | | | | | |
| GSRTGR001 | | | | | | | | | | |
| APD | 50.4 | 0.1 | 68.7 | 18.4 | 52.3 | 1.9 | 60.5 | 10.2 | 59.5 | 9.2 |
| Makeup | | | | | | | | | | |
| Covergirl | | | | | | | | | | |
| CW120 | | | | | | | | | | |
| GSRTCGM001 | | | | | | | | | | |
| APD Red | 39.9 | 9.0 | 56.7 | 25.9 | 31.6 | 0.7 | 47.0 | 16.1 | 39.8 | 8.9 |
| Wine | | | | | | | | | | |
| Wolf | | | | | | | | | | |
| Blas | | | | | | | | | | |
| CW120 | | | | | | | | | | |
| GSRTRW001 | | | | | | | | | | |
| APD | 32.3 | 2.2 | 33.5 | 3.4 | 32.7 | 2.7 | 34.5 | 4.4 | 34.9 | 4.8 |
| Spaghetti | | | | | | | | | | |
| Sauce | | | | | | | | | | |
| Ragu | | | | | | | | | | |
| CW120 | | | | | | | | | | |
| GSRTSS001 | | | | | | | | | | |
| APD Tea | 72.2 | 2.1 | 72.9 | 2.8 | 71.0 | 0.8 | 73.0 | 2.8 | 72.7 | 2.5 |
| American | | | | | | | | | | |
| Lipton | | | | | | | | | | |
| CW120 | | | | | | | | | | |
| GSRTLIT001 | | | | | | | | | | |
| CFT | 10.6 | 2.2 | 11.5 | 3.2 | 10.6 | 2.3 | 12.1 | 3.8 | 12.2 | 3.9 |
| ASTM | | | | | | | | | | |
| Dust | | | | | | | | | | |
| Sebum | | | | | | | | | | |
| PCS94 | | | | | | | | | | |
| CFT | 19.8 | 18.2 | 50.1 | 48.6 | 2.8 | 1.3 | 30.9 | 29.3 | 30.9 | 29.4 |
| Choc | | | | | | | | | | |
| Soy Milk | | | | | | | | | | |
| CS45 | | | | | | | | | | |
| CFT | 25.6 | 2.9 | 31.4 | 8.7 | 26.1 | 3.4 | 28.9 | 6.2 | 28.3 | 5.7 |
| Discriminating | | | | | | | | | | |
| Sebum | | | | | | | | | | |
| PCS132 | | | | | | | | | | |
| CFT | 29.1 | 10.7 | 48.3 | 29.8 | 21.3 | 2.9 | 30.9 | 12.5 | 28.9 | 10.5 |
| Rice | | | | | | | | | | |
| Starch | | | | | | | | | | |
| CS28 | | | | | | | | | | |
| | 68.2 | −2.1 | 72.6 | 2.3 | 71.3 | 1.0 | 71.5 | 1.2 | 71.8 | 1.5 |

C16 GS was superior to SLS at 200 ppm and 400 ppm in these tests.

### EXAMPLES

### Example 1: Sodium (2R,3R,4S,5R)-6-(dodecylamino)-2,3,4,5-tetrahydroxy-6-oxohexyl sulfate

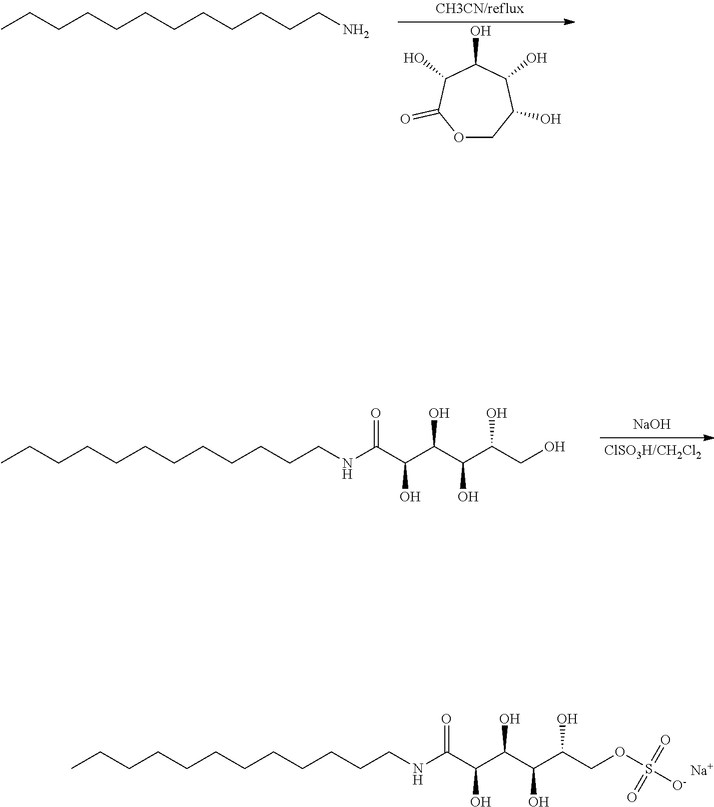 [SMILES: CCCCCCCCCCCCN ; O=C1OC[C@@H](O)[C@@H](O)[C@H](O)[C@H]1O ; CCCCCCCCCCCCNC(=O)[C@H](O)[C@@H](O)[C@H](O)[C@H](O)CO ; "NaOH" ; CCCCCCCCCCCCNC(=O)[C@H](O)[C@@H](O)[C@H](O)[C@H](O)COS(=O)(=O)[O-] ; [Na+]]

To a 1000-ml, single neck, round bottom reaction flask equipped with a magnetic stir bar was added 26 grams (0.141 moles) of Dodecyl amine (CAS 124-22-1/Sigma Aldrich), in 500 mL acetonitrile at room temperature. Once the solution was homogeneous, 24.7 grams gluconolactone (CAS 90-80-2, 0.140 mol) was added portion wise over 15 minutes. Attached to the reaction flask was a water-cooled condenser equipped with a nitrogen line at the top leading to a gas bubbler with mixing under a nitrogen atmosphere, the reaction flask was heated for 3 hours at reflux using a heating mantle, during which time the solid dissolved and the reaction became homogeneous. The reaction cooled to room temperature; at which time a precipitate could be seen forming. The reaction was then cooled to 0° C. and was allowed to stand 3 hours. The resulting precipitate was collected via filtration with course glass-sintered filter, wash 3×500 mL acetone, then dried under house vacuum (˜ 10 torr) for 24 hours at room temperature. The resulting white solid was weighed (46 grams recovery) and then used in the sulphation step without further purification.

To a 500-ml, single neck, round bottom reaction flask was added 21.378 grams (0.058813 moles) of N-Dodecyl-D-Gluconamide, 205 ml of ACS Reagent Grade Dichloromethane (EMD Millipore product #DX0835) and a magnetic stir bar. Attached to the reaction flask a water-cooled condenser with a dry nitrogen line attached to the top of condenser leading from a gas bubbler. Under nitrogen atmosphere, vigorously stirred reaction mixture while mildly heating reaction flask with an oil bath until Dichloromethane began to very mildly reflux on walls of reaction flask. After 35 minutes, the solid N-Dodecyl-D-Gluconamide was well dispersed as fine solid particles. Removed condenser from reaction flask and attached an addition funnel with pressure equalizing arm and nitrogen line which contained a solution of 6.876 grams (0.05901 moles) of 99+% Chlorosulfonic Acid (Sigma-Aldrich Product #571024) dissolved in 20 ml of ACS Reagent Grade Dichloromethane. With continued vigorous stirring and very mild reflux heating under nitrogen atmosphere, the Chlorosulfonic Acid/Dichloromethane solution was dripped into reaction mixture over a 17 minute period at which point the addition funnel was removed and a water-cooled condenser with nitrogen line was reattached to reaction flask. Reaction mixture was still heterogeneous with dispersed solid particles. Continued to stir vigorously under nitrogen atmosphere with mild reflux heating for 4 hours then let stir overnight at room temperature (21° C.) at which point reaction mixture was still heterogeneous with well dispersed solid particles. The condenser was removed, and a dry nitrogen gas flow was blown into the reaction flask while mixing and heating with a 25° C. oil bath to evaporate Dichloromethane and residual HCl which was generated during reaction. Once reaction mixture volume was reduced by about half, evaporation was taken to completeness using a rotary evaporator with water bath set at 40° C. yielding a white solid.

To the resulting solid was added a solution of 4.721 g of 50 wt % Sodium Hydroxide Reagent (0.0590 moles Sodium Hydroxide) dissolved in a mixture of 155 ml Deionized Water and 155 ml Absolute Ethanol followed by stirring to obtain a nearly clear, colorless solution with a small amount of insoluble crystalline solids. Vacuum filtered solution through Grade 4 filter paper to obtain a clear, colorless solution. This solution was measured to be pH 6.8 using a pH meter. Adjusted pH of the solution to 7.2 with the addition of 0.1 N Sodium Hydroxide. The solution was concentrated using a rotary evaporator until mixture began to foam, then solution was transferred to a glass crystallizing dish and placed in a vacuum oven at room temperature under a partial vacuum of 3 inches of Hg internal oven pressure. The next day, the product was removed from vacuum oven and found to be a partial solid. Mixed with a spatula and placed back into vacuum oven under partial vacuum with heating at 35° C. and removing from vacuum oven every 1-2 hours to mix followed with continue partial vacuum treatment and finally, once product was substantially a solid, placed under full vacuum at room temperature for 4 days yielding a crisp white solid. This solid was ground into a fine powder using a mortar and pestle then placed overnight in a vacuum oven under full vacuum at room temperature yielding 26.6 g of final product as a white solid powder. 0.0130 g of final product was mixed with 1.287 g of Deionized Water at room temperature to form a completely clear, colorless solution that foamed when shaken.

### Example 2: Sodium (2R,3R,4S,5R)-6-(tetradecylamino)-2,3,4,5-tetrahydroxy-6-oxohexyl sulfate

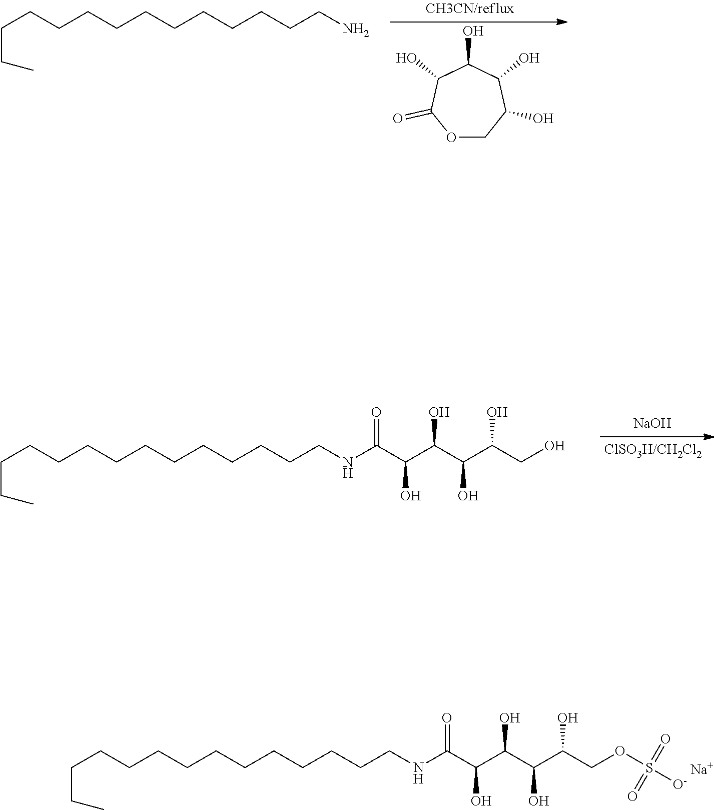 [SMILES: CCCCCCCCCCCCCCN ; O=C1OC[C@@H](O)[C@@H](O)[C@H](O)[C@H]1O ; CCCCCCCCCCCCCCNC(=O)[C@H](O)[C@@H](O)[C@H](O)[C@H](O)CO ; "NaOH" ; CCCCCCCCCCCCCCNC(=O)[C@H](O)[C@@H](O)[C@H](O)[C@H](O)COS(=O)(=O)[O-] ; [Na+]]

To a 1000-ml, single neck, round bottom reaction flask equipped with a magnetic stir bar was added 25 grams (0.117 moles) of Tetradecyl amine (CAS #2016-42-4/Sigma Aldrich), in 500 mL acetonitrile at room temperature. Once the solution was homogeneous, 20.1 grams (0.113 mol) gluconolactone (CAS 90-80-2, Sigma Aldrich) was added portion wise over 15 minutes. Attached to the reaction flask was a water-cooled condenser equipped with a nitrogen line at the top leading to a gas bubbler. With mixing under a nitrogen atmosphere, the reaction flask was heated for 3 hours at reflux using a heating mantle, during which time the solid dissolved and the reaction became homogeneous. The reaction cooled to room temperature; at which time a precipitate could be seen forming. The reaction was then cooled to 0° C. and was allowed to stand 3 hours. The resulting precipitate was collected via filtration with course glass-sintered filter, wash 3×500 mL acetone, then dried under house vacuum (˜ 10 torr) for 24 hours at room temperature. The resulting white solid was weighed (34.75 grams recovery) and then used in the sulphation step without further purification.

To a 500-ml, single neck, round bottom reaction flask was added 23.0 grams (0.058813 moles) of N-Tetradecyl-D-Gluconamide, 205 ml of ACS Reagent Grade Dichloromethane (EMD Millipore product #DX0835) and a magnetic stir bar. Attached to the reaction flask a water-cooled condenser with a dry nitrogen line attached to the top of condenser leading from a gas bubbler. Under nitrogen atmosphere, vigorously stirred reaction mixture while mildly heating reaction flask with an oil bath until Dichloromethane began to very mildly reflux on walls of reaction flask. After 35 minutes, the solid N-Tetradecyl-D-Gluconamide was well dispersed as fine solid particles. Removed condenser from reaction flask and attached an addition funnel with pressure equalizing arm and nitrogen line which contained a solution of 6.876 grams (0.05901 moles) of 99+% Chlorosulfonic Acid (Sigma-Aldrich Product #571024) dissolved in 20 ml of ACS Reagent Grade Dichloromethane. With continued vigorous stirring and very mild reflux heating under nitrogen atmosphere, the Chlorosulfonic Acid/Dichloromethane solution was dripped into reaction mixture over a 17 minute period at which point the addition funnel was removed and a water-cooled condenser with nitrogen line was reattached to reaction flask. Reaction mixture was still heterogeneous with dispersed solid particles. Continued to stir vigorously under nitrogen atmosphere with mild reflux heating for 4 hours then let stir overnight at room temperature (21° C.) at which point reaction mixture was still heterogeneous with well dispersed solid particles. The condenser was removed, and a dry nitrogen gas flow was blown into the reaction flask while mixing and heating with a 25° C. oil bath to evaporate Dichloromethane and residual HCl which was generated during reaction. Once reaction mixture volume was reduced by about half, evaporation was taken to completeness using a rotary evaporator with water bath set at 40° C. yielding a white solid.

To the resulting solid was added a solution of 4.721 g of 50 wt % Sodium Hydroxide Reagent (0.0590 moles Sodium Hydroxide) dissolved in a mixture of 155 ml Deionized Water and 155 ml Absolute Ethanol followed by stirring to obtain a nearly clear, colorless solution with a small amount of insoluble crystalline solids. Vacuum filtered solution through Grade 4 filter paper to obtain a clear, colorless solution. This solution was measured to be pH 6.8 using a pH meter. Adjusted pH of the solution to 7.2 with the addition of 0.1 N Sodium Hydroxide. The solution was concentrated using a rotary evaporator until mixture began to foam, then solution was transferred to a glass crystallizing dish and placed in a vacuum oven at room temperature under a partial vacuum of 3 inches of Hg internal oven pressure. The next day, the product was removed from vacuum oven and found to be a partial solid. Mixed with a spatula and placed back into vacuum oven under partial vacuum with heating at 35° C. and removing from vacuum oven every 1-2 hours to mix followed with continue partial vacuum treatment and finally, once product was substantially a solid, placed under full vacuum at room temperature for 4 days yielding a crisp white solid. This solid was ground into a fine powder using a mortar and pestle then placed overnight in a vacuum oven under full vacuum at room temperature yielding 22.8 g of final product as a white solid powder. 0.0130 g of final product was mixed with 1.287 g of Deionized Water at room temperature to form a completely clear, colorless solution that foamed when shaken.

### Example 3: Sodium (2R,3R,4S,5R)-6-(hexadecylamino)-2,3,4,5-tetrahydroxy-6-oxohexyl sulfate

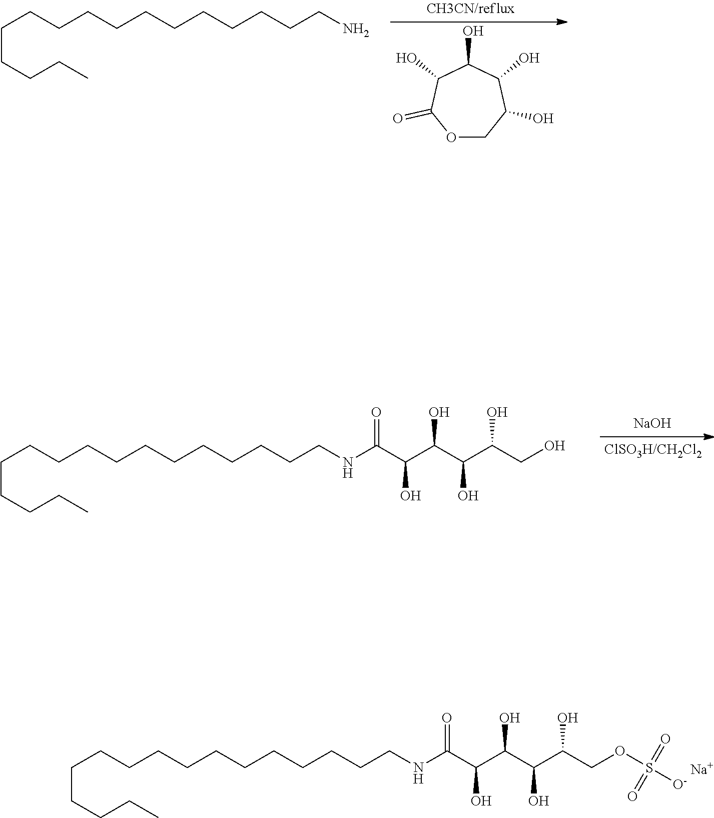 [SMILES: CCCCCCCCCCCCCCCCN ; O=C1OC[C@@H](O)[C@@H](O)[C@H](O)[C@H]1O ; CCCCCCCCCCCCCCCCNC(=O)[C@H](O)[C@@H](O)[C@H](O)[C@H](O)CO ; "NaOH" ; CCCCCCCCCCCCCCCCNC(=O)[C@H](O)[C@@H](O)[C@H](O)[C@H](O)COS(=O)(=O)[O-] ; [Na+]]

To a 1000-ml, single neck, round bottom reaction flask equipped with a magnetic stir bar was added 25 grams (0.103 mol) of Hexadecyl amine (CAS #143-27-1/Sigma Aldrich), in 500 mL acetonitrile at room temperature. Once the solution was homogeneous, 19.3 grams (0.105 mol) gluconolactone (CAS 90-80-2, Sigma Aldrich) was added portion wise over 15 minutes. Attached to the reaction flask was a water-cooled condenser equipped with a nitrogen line at the top leading to a gas bubbler Using mixing under a nitrogen atmosphere, the reaction flask was heated for 3 hours at reflux using a heating mantle, during which time the solid dissolved and the reaction became homogeneous. The reaction cooled to room temperature; at which time a precipitate could be seen forming. The reaction was then cooled to 0° C. and was allowed to stand 3 hours. The resulting precipitate was collected via filtration with course glass-sintered filter, wash 3×500 mL acetone, then dried under house vacuum (˜ 10 torr) for 24 hours at room temperature. The resulting white solid was weighed (36.64 grams recovery) and then used in the sulphation step without further purification.

To a 500-ml, single neck, round bottom reaction flask was added 24.64 grams (0.058813 moles) of N-Hexadecyl-D-Gluconamide, 205 ml of ACS Reagent Grade Dichloromethanc (EMD Millipore product #DX0835) and a magnetic stir bar. Attached to the reaction flask a water-cooled condenser with a dry nitrogen line attached to the top of condenser leading from a gas bubbler. Under nitrogen atmosphere, vigorously stirred reaction mixture while mildly heating reaction flask with an oil bath until Dichloromethane began to very mildly reflux on walls of reaction flask. After 35 minutes, the solid N-Hexadecyl-D-Gluconamide was well dispersed as fine solid particles. Removed condenser from reaction flask and attached an addition funnel with pressure equalizing arm and nitrogen line which contained a solution of 6.876 grams (0.05901 moles) of 99+% Chlorosulfonic Acid (Sigma-Aldrich Product #571024) dissolved in 20 ml of ACS Reagent Grade Dichloromethane. With continued vigorous stirring and very mild reflux heating under nitrogen atmosphere, the Chlorosulfonic Acid/Dichloromethane solution was dripped into reaction mixture over a 17 minute period at which point the addition funnel was removed and a water-cooled condenser with nitrogen line was reattached to reaction flask. Reaction mixture was still heterogeneous with dispersed solid particles. Continued to stir vigorously under nitrogen atmosphere with mild reflux heating for 4 hours then let stir overnight at room temperature (21° C.) at which point reaction mixture was still heterogeneous with well dispersed solid particles. The condenser was removed, and a dry nitrogen gas flow was blown into the reaction flask while mixing and heating with a 25° C. oil bath to evaporate Dichloromethane and residual HCl which was generated during reaction. Once reaction mixture volume was reduced by about half, evaporation was taken to completeness using a rotary evaporator with water bath set at 40° C. yielding a white solid.

To the resulting solid was added a solution of 4.721 g of 50 wt % Sodium Hydroxide Reagent (0.0590 moles Sodium Hydroxide) dissolved in a mixture of 155 ml Deionized Water and 155 ml Absolute Ethanol followed by stirring to obtain a nearly clear, colorless solution with a small amount of insoluble crystalline solids. Vacuum filtered solution through Grade 4 filter paper to obtain a clear, colorless solution. This solution was measured to be pH 6.8 using a pH meter. Adjusted pH of the solution to 7.2 with the addition of 0.1 N Sodium Hydroxide. The solution was concentrated using a rotary evaporator until mixture began to foam, then solution was transferred to a glass crystallizing dish and placed in a vacuum oven at room temperature under a partial vacuum of 3 inches of Hg internal oven pressure. The next day, the product was removed from vacuum oven and found to be a partial solid. Mixed with a spatula and placed back into vacuum oven under partial vacuum with heating at 35° C. and removing from vacuum oven every 1-2 hours to mix followed with continue partial vacuum treatment and finally, once product was substantially a solid, placed under full vacuum at room temperature for 4 days yielding a crisp white solid. This solid was ground into a fine powder using a mortar and pestle then placed overnight in a vacuum oven under full vacuum at room temperature yielding 26.6 g of final product as a white solid powder. 0.0130 g of final product was mixed with 1.287 g of Deionized Water at room temperature to form a completely clear, colorless solution that foamed when shaken.

### Example 4: Sodium (2R,3R,4S,5R)-2,3,4,5-tetrahydroxy-6-(((Z)-octadec-9-en-1-yl)amino)-6-oxohexyl sulfate

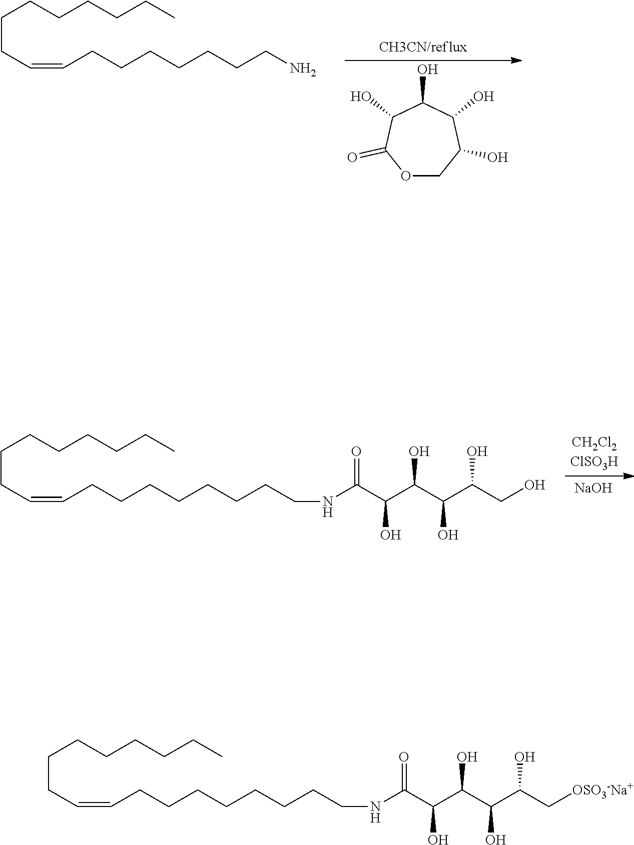

To a 500-ml, single neck, round bottom reaction flask equipped with a magnetic stir bar was added 10 grams (0.039moles) of Oleyl amine (112-90-3/Sigma Aldrich), in 300 mL acetonitrile at room temperature. Once the solution was homogeneous, 6.6 grams gluconolactone (CAS 90-80-2, 0.037 mol) was added portion wise over 15 minutes. Attached to the reaction flask was a water-cooled condenser equipped with a nitrogen line at the top leading to a gas bubbler. With mixing under a nitrogen atmosphere, the reaction flask was heated for 3 hours at reflux using a heating mantle, during which time the solid dissolved and the reaction became homogeneous. The reaction cooled to room temperature; at which time a precipitate could be seen forming. The reaction was then cooled to 0° C. and was allowed to stand 3 hours. The resulting precipitate was collected via filtration with course glass-sintered filter, wash 3×300 mL acetone, then dried under house vacuum (˜ 10 torr) for 24 hours at room temperature. The resulting white solid was weighed 15.3 grams after recovery.

To a 500-ml, single neck, round bottom reaction flask was added 26.18 grams (0.058813 moles) of N-Oleyl-D-Gluconamide, 205 ml of ACS Reagent Grade Dichloromethane (EMD Millipore product #DX0835) and a magnetic stir bar. Attached to the reaction flask a water-cooled condenser with a dry nitrogen line attached to the top of condenser leading from a gas bubbler. Under nitrogen atmosphere, vigorously stirred reaction mixture while mildly heating reaction flask with an oil bath until Dichloromethane began to very mildly reflux on walls of reaction flask. After 35 minutes, the solid N-Oleyl-D-Gluconamide was well dispersed as fine solid particles. Removed condenser from reaction flask and attached an addition funnel with pressure equalizing arm and nitrogen line which contained a solution of 6.876 grams (0.05901 moles) of 99+% Chlorosulfonic Acid (Sigma-Aldrich Product #571024) dissolved in 20 ml of ACS Reagent Grade Dichloromethane. With continued vigorous stirring and very mild reflux heating under nitrogen atmosphere, the Chlorosulfonic Acid/Dichloromethane solution was dripped into reaction mixture over a 17 minute period at which point the addition funnel was removed and a water-cooled condenser with nitrogen line was reattached to reaction flask. Reaction mixture was still heterogeneous with dispersed solid particles. Continued to stir vigorously under nitrogen atmosphere with mild reflux heating for 4 hours then let stir overnight at room temperature (21° C.) at which point reaction mixture was still heterogeneous with well dispersed solid particles. The condenser was removed, and a dry nitrogen gas flow was blown into the reaction flask while mixing and heating with a 25° C. oil bath to evaporate Dichloromethane and residual HCl which was generated during reaction. Once reaction mixture volume was reduced by about half, evaporation was taken to completeness using a rotary evaporator with water bath set at 40° C. yielding a white solid.

To the resulting solid was added a solution of 4.721 g of 50 wt % Sodium Hydroxide Reagent (0.0590 moles Sodium Hydroxide) dissolved in a mixture of 155 ml Deionized Water and 155 ml Absolute Ethanol followed by stirring to obtain a nearly clear, colorless solution with a small amount of insoluble crystalline solids. Vacuum filtered solution through Grade 4 filter paper to obtain a clear, colorless solution. This solution was measured to be pH 6.8 using a pH meter. Adjusted pH of the solution to 7.2 with the addition of 0.1 N Sodium Hydroxide. The solution was concentrated using a rotary evaporator until mixture began to foam, then solution was transferred to a glass crystallizing dish and placed in a vacuum oven at room temperature under a partial vacuum of 3 inches of Hg internal oven pressure. The next day, the product was removed from vacuum oven and found to be a partial solid. Mixed with a spatula and placed back into vacuum oven under partial vacuum with heating at 35° C. and removing from vacuum oven every 1-2 hours to mix followed with continue partial vacuum treatment and finally, once product was substantially a solid, placed under full vacuum at room temperature for 4 days yielding a crisp white solid. This solid was ground into a fine powder using a mortar and pestle then placed overnight in a vacuum oven under full vacuum at room temperature yielding 26.6 g of final product as a white solid powder. 0.0130 g of final product was mixed with 1.287 g of Deionized Water at room temperature to form a completely clear, colorless solution that foamed when shaken.

### Example 5: Sodium (2R,3R,4S,5R)-6-((3-(butyl(pentyl)amino) propyl)amino)-2,3,4,5-tetrahydroxy-6-oxohexyl sulfate

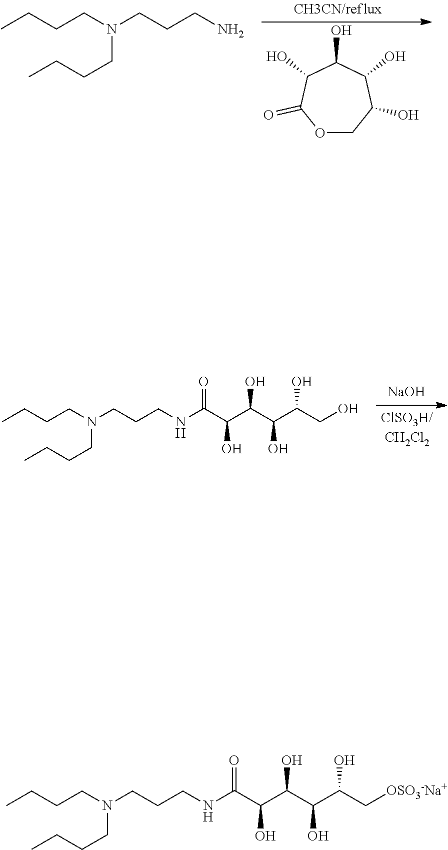

To a 1000-ml, single neck, round bottom reaction flask equipped with a magnetic stir bar was added 25 grams (0.134 mol) of Dibutylpropylamine (CAS102-83-0, Sigma Aldrich), in 500 mL acetonitrile at room temperature. Once the solution was homogeneous, 21.5 grams gluconolactone (CAS 90-80-2, 0.121 mols) was added portion wise over 15 minutes. Attached to the reaction flask was a water-cooled condenser equipped with a nitrogen line at the top leading to a gas bubbler. With mixing under a nitrogen atmosphere, the reaction flask was heated for 3 hours at reflux using a heating mantle, during which time the solid dissolved and the reaction became homogeneous. The reaction cooled to room temperature; at which time a precipitate could be seen forming. The reaction was then cooled to 0° C. and was allowed to stand 3 hours. The resulting precipitate was collected via filtration with course glass-sintered filter, wash 3×500 mL acetone, then dried under house vacuum (˜10 torr) for 24 hours at room temperature. The resulting white solid was weighed (40.3 grams recovery) and then used in the sulphation step without further purification.

To a 500 ml, single neck, round bottom reaction flask was added 24.41 grams (0.058813 moles) of N-Dibutylpropyl-D-Gluconamide, 205 ml of ACS Reagent Grade Dichloromethane (EMD Millipore product #DX0835) and a magnetic stir bar. Attached to the reaction flask a water-cooled condenser with a dry nitrogen line attached to the top of condenser leading from a gas bubbler. Under nitrogen atmosphere, vigorously stirred reaction mixture while mildly heating reaction flask with an oil bath until Dichloromethane began to very mildly reflux on walls of reaction flask. After 35 minutes, the solid N-Dibutylpropyl-D-Gluconamide was well dispersed as fine solid particles. Removed condenser from reaction flask and attached an addition funnel with pressure equalizing arm and nitrogen line which contained a solution of 6.876 grams (0.05901 moles) of 99+% Chlorosulfonic Acid (Sigma-Aldrich Product #571024) dissolved in 20 ml of ACS Reagent Grade Dichloromethane. With continued vigorous stirring and very mild reflux heating under nitrogen atmosphere, the Chlorosulfonic Acid/Dichloromethane solution was dripped into reaction mixture over a 17 minute period at which point the addition funnel was removed and a water-cooled condenser with nitrogen line was reattached to reaction flask. Reaction mixture was still heterogeneous with dispersed solid particles. Continued to stir vigorously under nitrogen atmosphere with mild reflux heating for 4 hours then let stir overnight at room temperature (21° C.) at which point reaction mixture was still heterogeneous with well dispersed solid particles. The condenser was removed, and a dry nitrogen gas flow was blown into the reaction flask while mixing and heating with a 25° C. oil bath to evaporate Dichloromethane and residual HCl which was generated during reaction. Once reaction mixture volume was reduced by about half, evaporation was taken to completeness using a rotary evaporator with water bath set at 40° C. yielding a white solid.

To the resulting solid was added a solution of 4.721 g of 50 wt % Sodium Hydroxide Reagent (0.0590 moles Sodium Hydroxide) dissolved in a mixture of 155 ml Deionized Water and 155 ml Absolute Ethanol followed by stirring to obtain a nearly clear, colorless solution with a small amount of insoluble crystalline solids. Vacuum filtered solution through Grade 4 filter paper to obtain a clear, colorless solution. This solution was measured to be pH 6.8 using a pH meter. Adjusted pH of the solution to 7.2 with the addition of 0.1 N Sodium Hydroxide. The solution was concentrated using a rotary evaporator until mixture began to foam, then solution was transferred to a glass crystallizing dish and placed in a vacuum oven at room temperature under a partial vacuum of 3 inches of Hg internal oven pressure. The next day, the product was removed from vacuum oven and found to be a partial solid. Mixed with a spatula and placed back into vacuum oven under partial vacuum with heating at 35° C. and removing from vacuum oven every 1-2 hours to mix followed with continue partial vacuum treatment and finally, once product was substantially a solid, placed under full vacuum at room temperature for 4 days yielding a crisp white solid. This solid was ground into a fine powder using a mortar and pestle then placed overnight in a vacuum oven under full vacuum at room temperature yielding 26.6 g of final product as a white solid powder. 0.0130 g of final product was mixed with 1.287 g of Deionized Water at room temperature to form a completely clear, colorless solution that foamed when shaken.

### Example 6: Sodium (2R,3R,4S,5R)-6-((3-dodecyloxy) propyl)amino)-2,3,4,5-tetrahydroxy-6-oxohexyl sulfate

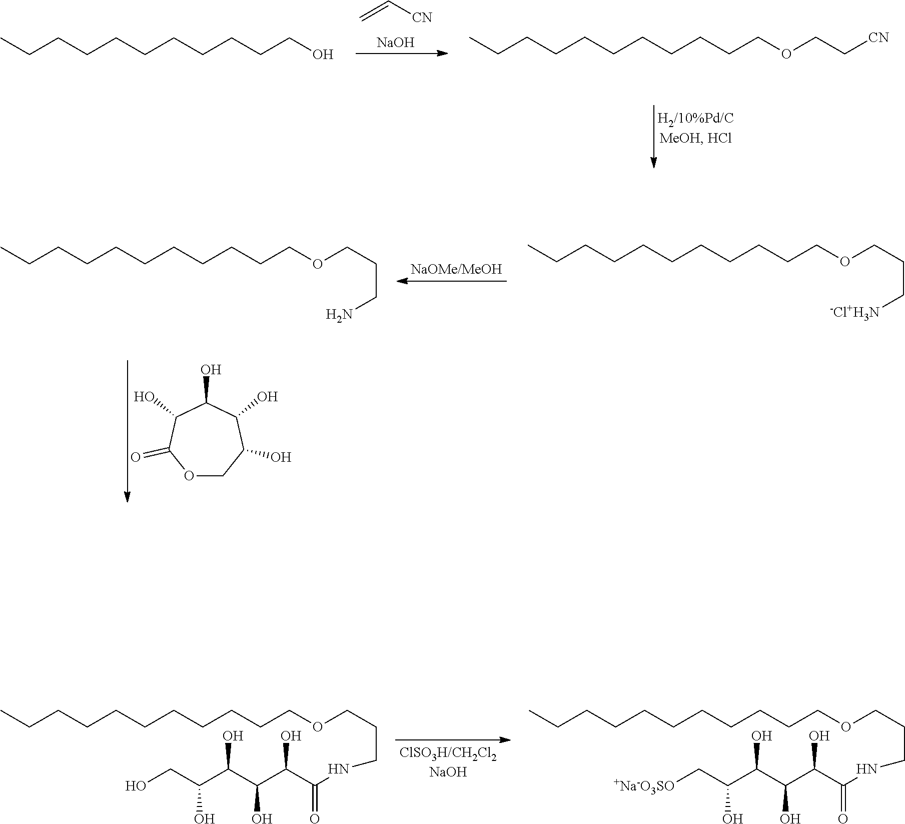

### Step 1:3-(Dodecyloxy) Propanenitrile

Lauryl alcohol (86.461 g, 0.464 mol) was placed in a 250 mL RBF. The flask was heated at 65° C. Crushed NaOH (0.371 g, 2 mol %) was added and the mixture was stirred for 1 hr to give a clear solution. Acrylonitrile (31.94 mL, 1.05 eq.) was added slowly in 94 min via a syringe pump with its needle submerging under the liquid surface. After the addition, the hazy solution was stirred at 65° C. for 2 hrs. The oil bath was removed and the mixture was cooled to room temperature (RT) and kept under high vacuum overnight. The content was filtered to remove insoluble materials and the filtrate (112.03 g) was clear and light yellow oil. ¹HNMR (Proton NMR) analysis of the crude product indicated about 97% conversion of the starting material to the product. The filtrate was used for the next step without purification.

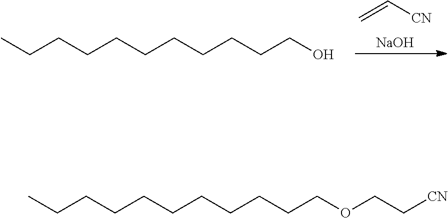

### Step 2:3-(Dodecyloxy) Propan-1-Amine Hydrochloride

3-(Dodecyloxy) propanenitrile (112.03 g) was dissolved in MeOH (1.0 L) in a 3-L pressurized glass vessel, 12N HCl (38.67 mL, 1 eq.) was added followed by 10% Pd/C (44.8 g, 40 w/w %). The mixture was purged with hydrogen 3 times and kept at 40 psi of H₂ for 7 days. The hydrogen pressure was adjusted to 40 psi daily and the reaction was monitored by Thin Layer Chromatography (TLC) analysis. At the end of the hydrogenation, the reaction vessel was purged with nitrogen 3 times. The solid was removed by filtration, the filtrate was evaporated on a rotary evaporator and further dried under high vacuum overnight. The solid was suspended in EtOAc (800 mL) and triturated at RT overnight. The solid was collected by filtration and further dried under high vacuum to give 99.95 g (77% for 2 steps) of a white solid.

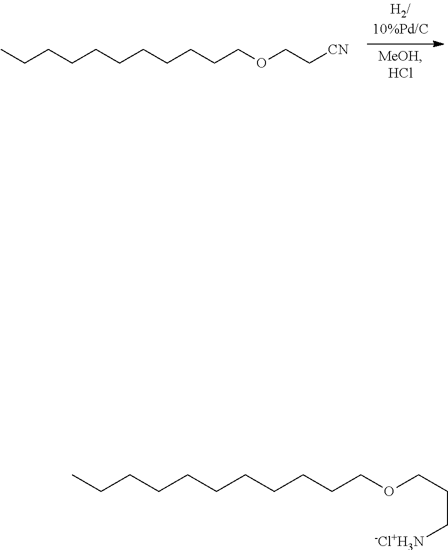

### Step 3:3-(Dodecyloxy) propan-1-amine

To a suspension of MeOH (100 mL) and 30 g of HCl salt above (0.107 mol) was added 258 mL (>0.5 M in MeOH/.129 mol) of sodium methoxide dropwise over 20 minutes. No exotherm was observed. The reaction was stirred 24 at RT, then filtered to remove precipitate. The mother liquor was evaporated (rotovap) and the residue was partitioned between Et2O and IN NaOH. The layers were separated and the organic was washed with brine, dried (Na2SO4) and concentrated to provide the amine as a white liquid. 23 grams was isolated and used in next step.

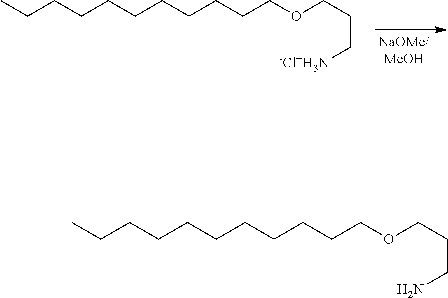

### Step 4: (2R,3R,4S,5R)-2,3,4,5,6-pentahydroxy-N-(3-(Undecyloxy) Propyl) Hexanamide

To the amine in CH3CN (200 mL) was added gluconolactone and the reaction was refluxed 6 hr. The reaction was cooled to RT, and the product filtered off as a white solid. The product was suspended in hot acetone, stirred 20 minutes, then filtered. The product was dried 24 h under vacuum on house vac (5-10 mm Hg). Yield: 31.5 g (79%)

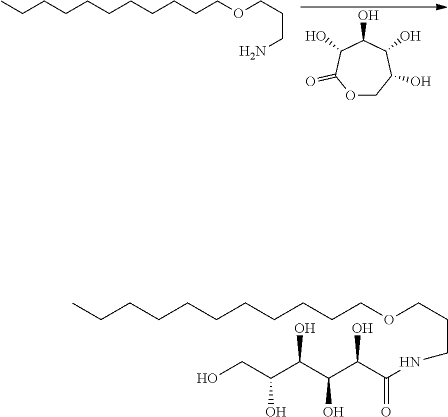

### Step 5: Sodium (2R,3R,4S,5R)-6-((3-dodecyloxy) propyl)amino)-2,3,4,5-tetrahydroxy-6-Oxohexyl Sulfate

To a 500-ml, single neck, round bottom reaction flask was added 23.93 grams (0.058813 moles) of (2R,3R,4S,5R)-2,3,4,5,6-pentahydroxy-N-(3-(undecyloxy) propyl) hexanamide, 205 ml of ACS Reagent Grade Dichloromethane (EMD Millipore product #DX0835) and a magnetic stir bar. Attached to the reaction flask a water-cooled condenser with a dry nitrogen line attached to the top of condenser leading from a gas bubbler. Under nitrogen atmosphere, vigorously stirred reaction mixture while mildly heating reaction flask with an oil bath until Dichloromethane began to very mildly reflux on walls of reaction flask. After 35 minutes, the solid hexanamide was well dispersed as fine solid particles. Removed condenser from reaction flask and attached an addition funnel with pressure equalizing arm and nitrogen line which contained a solution of 6.876 grams (0.05901 moles) of 99+% Chlorosulfonic Acid (Sigma-Aldrich Product #571024) dissolved in 20 ml of ACS Reagent Grade Dichloromethane. With continued vigorous stirring and very mild reflux heating under nitrogen atmosphere, the Chlorosulfonic Acid/Dichloromethane solution was dripped into reaction mixture over a 17 minute period at which point the addition funnel was removed and a water-cooled condenser with nitrogen line was reattached to reaction flask. Reaction mixture was still heterogeneous with dispersed solid particles. Continued to stir vigorously under nitrogen atmosphere with mild reflux heating for 4 hours then let stir overnight at room temperature (21° C.) at which point reaction mixture was still heterogeneous with well dispersed solid particles. The condenser was removed, and a dry nitrogen gas flow was blown into the reaction flask while mixing and heating with a 25° C. oil bath to evaporate Dichloromethane and residual HCl which was generated during reaction. Once reaction mixture volume was reduced by about half, evaporation was taken to completeness using a rotary evaporator with water bath set at 40° C. yielding a white solid.

To the resulting solid was added a solution of 4.721 g of 50 wt % Sodium Hydroxide Reagent (0.0590 moles Sodium Hydroxide) dissolved in a mixture of 155 ml Deionized Water and 155 ml Absolute Ethanol followed by stirring to obtain a nearly clear, colorless solution with a small amount of insoluble crystalline solids. Vacuum filtered solution through Grade 4 filter paper to obtain a clear, colorless solution. This solution was measured to be pH 6.8 using a pH meter. Adjusted pH of the solution to 7.2 with the addition of 0.1 N Sodium Hydroxide. The solution was concentrated using a rotary evaporator until mixture began to foam, then solution was transferred to a glass crystallizing dish and placed in a vacuum oven at room temperature under a partial vacuum of 3 inches of Hg internal oven pressure. The next day, the product was removed from vacuum oven and found to be a partial solid. Mixed with a spatula and placed back into vacuum oven under partial vacuum with heating at 35° C. and removing from vacuum oven every 1-2 hours to mix followed with continue partial vacuum treatment and finally, once product was substantially a solid, placed under full vacuum at room temperature for 4 days yielding a crisp white solid. This solid was ground into a fine powder using a mortar and pestle then placed overnight in a vacuum oven under full vacuum at room temperature yielding 20.5 grams of final product as a white solid powder. 0.0130 g of final product was mixed with 1.287 g of Deionized Water at room temperature to form a completely clear, colorless solution that foamed when shaken.

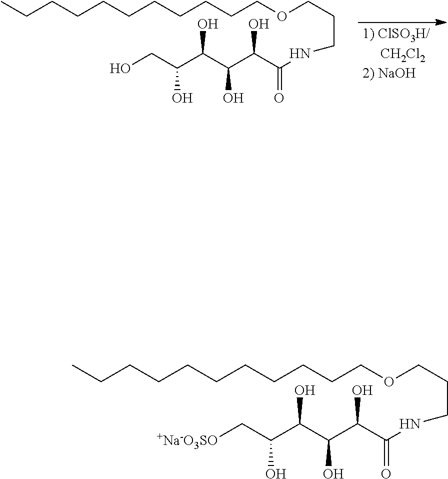

### Example 7: Sodium (2R,3R,4S,5R)-6-(benzylamino)-2,3,4,5-tetrahydroxy-6-oxohexyl Sulfate

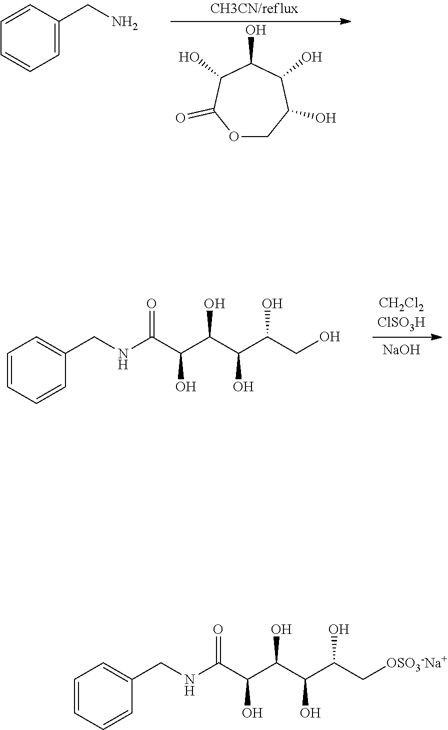

To a 500-ml, single neck, round bottom reaction flask equipped with a magnetic stir bar was added 10 grams (0.093 moles) of Benzyl amine (CAS 100-46-9/Sigma Aldrich), in 300 mL acetonitrile at room temperature. To this solution was added 15 grams gluconolactone (CAS 90-80-2, 0.085 mol) portion wise over 15 minutes. Attached to the reaction flask was a water-cooled condenser equipped with a nitrogen line at the top leading to a gas bubbler with mixing under a nitrogen atmosphere, the reaction flask was heated for 24 hours at reflux using a heating mantle, during which time the solid dissolved and the reaction became homogeneous. The reaction cooled to room temperature; at which time a precipitate could be seen forming. The reaction was then cooled to 0° C. and was allowed to stand 3 hours. The resulting precipitate was collected via filtration with course glass-sintered filter, washed 3×500 mL acetone, then dried under house vacuum (˜ 10 torr) for 24 hours at room temperature. The resulting white solid was weighed (22.6 grams recovery) and then used in the sulphation step without further purification.

To a 500-ml, single neck, round bottom reaction flask was added 16.76 grams (0.058813 moles) of N-Benzyl-D-Gluconamide, 205 ml of ACS Reagent Grade Dichloromethane (EMD Millipore product #DX0835) and a magnetic stir bar. Attached to the reaction flask a water-cooled condenser with a dry nitrogen line attached to the top of condenser leading from a gas bubbler. Under nitrogen atmosphere, vigorously stirred reaction mixture while mildly heating reaction flask with an oil bath until Dichloromethane began to very mildly reflux on walls of reaction flask. After 35 minutes, the solid N-Benzyl-D-Gluconamide was well dispersed as fine solid particles. Removed condenser from reaction flask and attached an addition funnel with pressure equalizing arm and nitrogen line which contained a solution of 6.876 grams (0.05901 moles) of 99+% Chlorosulfonic Acid (Sigma-Aldrich Product #571024) dissolved in 20 ml of ACS Reagent Grade Dichloromethane. With continued vigorous stirring and very mild reflux heating under nitrogen atmosphere, the Chlorosulfonic Acid/Dichloromethane solution was dripped into reaction mixture over a 17 minute period at which point the addition funnel was removed and a water-cooled condenser with nitrogen line was reattached to reaction flask. Reaction mixture was still heterogeneous with dispersed solid particles. Continued to stir vigorously under nitrogen atmosphere with mild reflux heating for 4 hours then let stir overnight at room temperature (21° C.) at which point reaction mixture was still heterogeneous with well dispersed solid particles. The condenser was removed, and a dry nitrogen gas flow was blown into the reaction flask while mixing and heating with a 25° C. oil bath to evaporate Dichloromethane and residual HCl which was generated during reaction. Once reaction mixture volume was reduced by about half, evaporation was taken to completeness using a rotary evaporator with water bath set at 40° C. yielding a white solid.

To the resulting solid was added a solution of 4.721 g of 50 wt % Sodium Hydroxide Reagent (0.0590 moles Sodium Hydroxide) dissolved in a mixture of 155 ml Deionized Water and 155 ml Absolute Ethanol followed by stirring to obtain a nearly clear, colorless solution with a small amount of insoluble crystalline solids. Vacuum filtered solution through Grade 4 filter paper to obtain a clear, colorless solution. This solution was measured to be pH 6.8 using a pH meter. Adjusted pH of the solution to 7.2 with the addition of 0.1 N Sodium Hydroxide. The solution was concentrated using a rotary evaporator until mixture began to foam, then solution was transferred to a glass crystallizing dish and placed in a vacuum oven at room temperature under a partial vacuum of 3 inches of Hg internal oven pressure. The next day, the product was removed from vacuum oven and found to be a partial solid. Mixed with a spatula and placed back into vacuum oven under partial vacuum with heating at 35° C. and removing from vacuum oven every 1-2 hours to mix followed with continue partial vacuum treatment and finally, once product was substantially a solid, placed under full vacuum at room temperature for 4 days yielding a crisp white solid. This solid was ground into a fine powder using a mortar and pestle then placed overnight in a vacuum oven under full vacuum at room temperature yielding 26.6 g of final product as a white solid powder. 0.0130 g of final product was mixed with 1.287 g of Deionized Water at room temperature to form a completely clear, colorless solution that foamed when shaken.

### Example 8: Sodium (2R,3R,4S,5R)-6-((2-1H-indol-2-yl)ethyl) amino)-2,3,4,5-tetrahydroxy-6-Oxohexyl Sulfate

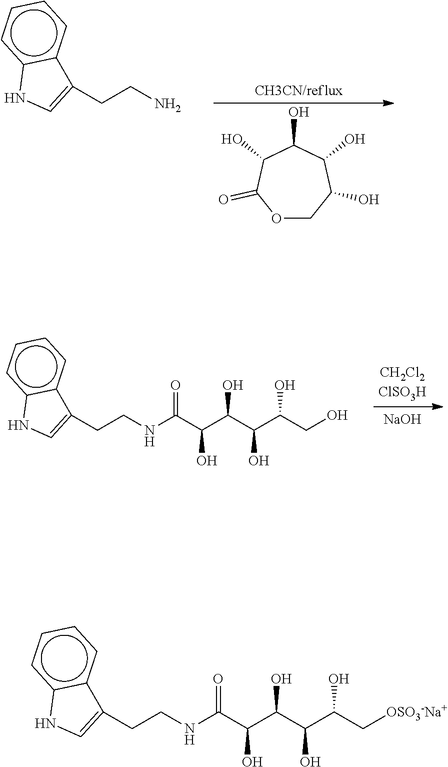

To a 500-ml, single neck, round bottom reaction flask equipped with a magnetic stir bar was added 10 grams (0.062 moles) of Tryptamine (CAS 61-54-1/Sigma Aldrich), in 300 mL acetonitrile at room temperature. Once the solution was homogeneous, 10.10 grams gluconolactone (CAS 90-80-2, 0.56moles) was added portion wise over 15 minutes. Attached to the reaction flask was a water-cooled condenser equipped with a nitrogen line at the top leading to a gas bubbler with mixing under a nitrogen atmosphere, the reaction flask was heated for 24 hours at reflux using a heating mantle, during which time the solid dissolved and the reaction became homogeneous. The reaction cooled to room temperature; at which time a precipitate could be seen forming. The reaction was then cooled to 0° C. and was allowed to stand 24 hours. The resulting precipitate was collected via filtration with course glass-sintered filter, wash 3×500 mL acetone, then dried under house vacuum (˜ 10 torr) for 24 hours at room temperature. The resulting white solid was weighed 16.9 grams after recovery.

To a 500-ml, single neck, round bottom reaction flask was added 19.88 grams (0.058813 moles) of N-Indolo-D-Gluconamide, 205 ml of ACS Reagent Grade Dichloromethane (EMD Millipore product #DX0835) and a magnetic stir bar. Attached to the reaction flask a water-cooled condenser with a dry nitrogen line attached to the top of condenser leading from a gas bubbler. Under nitrogen atmosphere, vigorously stirred reaction mixture while mildly heating reaction flask with an oil bath until Dichloromethane began to very mildly reflux on walls of reaction flask. After 35 minutes, the solid N-Indolo-D-Gluconamide was well dispersed as fine solid particles. Removed condenser from reaction flask and attached an addition funnel with pressure equalizing arm and nitrogen line which contained a solution of 6.876 grams (0.05901 moles) of 99+% Chlorosulfonic Acid (Sigma-Aldrich Product #571024) dissolved in 20 ml of ACS Reagent Grade Dichloromethane. With continued vigorous stirring and very mild reflux heating under nitrogen atmosphere, the Chlorosulfonic Acid/Dichloromethane solution was dripped into reaction mixture over a 17 minute period at which point the addition funnel was removed and a water-cooled condenser with nitrogen line was reattached to reaction flask. Reaction mixture was still heterogeneous with dispersed solid particles. Continued to stir vigorously under nitrogen atmosphere with mild reflux heating for 4 hours then let stir overnight at room temperature (21° C.) at which point reaction mixture was still heterogeneous with well dispersed solid particles. The condenser was removed, and a dry nitrogen gas flow was blown into the reaction flask while mixing and heating with a 25° C. oil bath to evaporate Dichloromethane and residual HCl which was generated during reaction. Once reaction mixture volume was reduced by about half, evaporation was taken to completeness using a rotary evaporator with water bath set at 40° C. yielding a white solid.

To the resulting solid was added a solution of 4.721 g of 50 wt % Sodium Hydroxide Reagent (0.0590 moles Sodium Hydroxide) dissolved in a mixture of 155 ml Deionized Water and 155 ml Absolute Ethanol followed by stirring to obtain a nearly clear, colorless solution with a small amount of insoluble crystalline solids. Vacuum filtered solution through Grade 4 filter paper to obtain a clear, colorless solution. This solution was measured to be pH 6.8 using a pH meter. Adjusted pH of the solution to 7.2 with the addition of 0.1 N Sodium Hydroxide. The solution was concentrated using a rotary evaporator until mixture began to foam, then solution was transferred to a glass crystallizing dish and placed in a vacuum oven at room temperature under a partial vacuum of 3 inches of Hg internal oven pressure. The next day, the product was removed from vacuum oven and found to be a partial solid. Mixed with a spatula and placed back into vacuum oven under partial vacuum with heating at 35° C. and removing from vacuum oven every 1-2 hours to mix followed with continue partial vacuum treatment and finally, once product was substantially a solid, placed under full vacuum at room temperature for 4 days yielding a crisp white solid. This solid was ground into a fine powder using a mortar and pestle then placed overnight in a vacuum oven under full vacuum at room temperature yielding 26.6 g of final product as a white solid powder. 0.0130 g of final product was mixed with 1.287 g of Deionized Water at room temperature to form a completely clear, colorless solution that foamed when shaken.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, comprising any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

The invention claimed is:

1. A compound comprising the following structure: 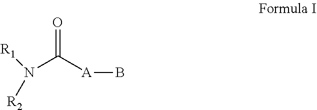
where:
A=polyhydroxylated alkyl, saccharide;
B=—OSO₃⁻M⁺, where M is a cation that forms an acceptable salt;
R₁=a heterocyclic, substituted or unsubstituted straight chain or branched chain heteroalkyl, alkyl or alkenyl, having 1 to 22 carbon and/or heteroatoms; and
R₂═H; alkyl;
including all stereoisomers thereof.

2. The compound of claim 1 comprising a sulfate, wherein the sulfate is at least one of the following:
sodium (2R,3R,4S,5R)-6-(docosylamino)-2,3,4,5-tetrahydroxy-6-oxohexyl sulfate;
sodium (2R,3R,4S,5R)-2,3,4,5-tetrahydroxy-6-(icosylamino)-6-oxohexyl sulfate;
sodium (2R,3R,4S,5R)-2,3,4,5-tetrahydroxy-6-(octadecylamino)-6-oxohexyl sulfate;
sodium (2R,3R,4S,5R)—N-Dodecyl-D-glycero-D-gulo-heptanamide;
sodium (2R,3R,4S,5R)-2,3,4,5-tetrahydroxy-6-(((Z)-octadec-9-en-1-yl)amino)-6-oxohexyl sulfate;
sodium (2R,3R,4S,5R)-6-(hexadecylamino)-2,3,4,5-tetrahydroxy-6-oxohexyl sulfate;
sodium (2R,3R,4S,5R)-6-(((E)-hexadec-7-en-1-yl)amino)-2,3,4,5-tetrahydroxy-6-oxohexyl sulfate;
sodium (2R,3R,4S,5R)-6-(tetradecylamino)-2,3,4,5-tetrahydroxy-6-oxohexyl sulfate;
sodium (2R,3R,4S,5R)—N-Dodecyl-D-glycero-D-gulo-heptanamide;
sodium (2R,3R,4S,5R)-2,3,4,5-tetrahydroxy-6-(undecylamino)-6-oxohexyl sulfate;
sodium (2R,3R,4S,5R)-2,3,4,5-tetrahydroxy-6-(decylamino)-6-oxohexyl sulfate;
sodium (2R,3R,4S,5R)-2,3,4,5-tetrahydroxy-6-(nonylamino)-6-oxohexyl sulfate;
sodium (2R,3R,4S,5R)-2,3,4,5-tetrahydroxy-6-(octylamino)-6-oxohexyl sulfate;
sodium (2R,3R,4S,5R)-6-((2-ethylhexyl)amino-2,3,4,5-tertahydroxy-6-oxohexyl sulfate;
sodium (2R,3R,4S,5R)-2,3,4-trihydroxy-5-(octylamino)-5-oxopentyl sulfate;
sodium (2R,3R,4R)-5-(heptylamino)-2,3,4-trihydroxy-5-oxopentyl sulfate;
sodium (2R,3R,4S,5R)-6-(hexylamino)-2,3,4,5-tetrahydroxy-6-oxohexyl sulfate;
sodium (2R,3R,4S,5S)-2,3,4,5-tetrahydroxy-6-oxo-6-(pentylamino) hexyl sulfate;
sodium (2R,3R,4S,5R)-6-(butylamino)-2,3,4,5-tetrahydroxy-6-oxohexyl sulfate;
sodium (2R,3R,4S,5R)-6-(sec-butylamino)-2,3,4,5-tetrahydroxy-6-oxohexyl sulfate;
sodium (2R,3R,4S,5R)-6-((2-1H-indol-2-yl)ethyl) amino)-2,3,4,5-tetrahydroxy-6-oxohexyl sulfate;
sodium (2R,3R,4S,5R)-2,3,4,5-tetrahydroxy-6-oxo-6 ((3-(tetradecylamino) propyl)amino) hexyl sulfate;
sodium (2R,3R,4S,5R)-6-((3-dodecylamino) propyl)amino)-2,3,4,5-tetrahydroxy-6-oxohexyl sulfate;
sodium (2R,3R,4S,5R)-6-((3-decylamino) propyl)amino)-2,3,4,5-tetrahydroxy-6-oxohexyl sulfate;
sodium (2R,3R,4S,5R)-2,3,4,5-tetrahydroxy-6-((2-(octylamino) propyl)amino)-6-oxohexyl sulfate;
sodium (2R,3R,4S,5R)-6-((3-(dibutylamino) propyl)amino)-2,3,4,5-tetrahydroxy-6-oxohexyl sulfate;
sodium (2R,3R,4R,5R)-2,3,4,5-tetrahydroxy-6-(((S)-2-methyl-5-pentanamidopentyl)amino)-6-oxohexyl sulfate;
sodium (2R,3R,4S,5R)-6-(((R)-5-hexanamido-4-methylpentyl)amino)-2,3,4,5-tetrahydroxy-6-oxohexyl sulfate:
sodium (2R,3R,4S,5R)-6-((3-((2-ethylhexyl)oxy) propyl)amino)-2,3,4,5-tetrahydroxy-6-oxohexyl sulfate;
sodium (2R,3R,4R)-5-((3-((2-ethylhexyl)oxy) propyl)amino)-2,3,4-trihydroxy-5-oxopentyl sulfate;
sodium (2R,3R,4S,5R,6R)-2,3,4,5,6-pentahydroxy-7-((3-hydroxypropyl)amino)-7-oxoheptyl sulfate;
sodium (2R,3R,4S,5R)-6-(benzylamino)-2,3,4,5-tetrahydroxy-6-oxohexyl sulfate;
and stereoisomers thereof, salt forms thereof, or mixtures thereof.

3. The compound of claim 1 comprising a sulfate, wherein the sulfate is at least one of:
sodium (2R,3R,4S,5R)-6-(hexadecylamino)-2,3,4,5-tetrahydroxy-6-oxohexyl sulfate;
sodium (2R,3R,4S,5R)-6-(((E)-hexadec-7-en-1-yl)amino)-2,3,4,5-tetrahydroxy-6-oxohexyl sulfate;
sodium (2R,3R,4S,5S)-2,3,4,5-tetrahydroxy-6-oxo-6-(pentylamino) hexyl sulfate;
sodium (2R,3R,4S,5R)-6-(butylamino)-2,3,4,5-tetrahydroxy-6-oxohexyl sulfate;
sodium (2R,3R,4S,5R)-6-(sec-butylamino)-2,3,4,5-tetrahydroxy-6-oxohexyl sulfate;
sodium (2R,3R,4S,5R)-6-(tetradecylamino)-2,3,4,5-tetrahydroxy-6-oxohexyl sulfate;
sodium (2R,3R,4S,5R)-6-(hexadecylamino)-2,3,4,5-tetrahydroxy-6-oxohexyl sulfate;
sodium (2R,3R,4S,5R)-2,3,4,5-tetrahydroxy-6-(((Z)-octadec-9-en-1-yl)amino)-6-oxohexyl sulfate;
sodium (2R,3R,4S,5R)-6-((3-(butyl(pentyl)amino) propyl)amino)-2,3,4,5-tetrahydroxy-6-oxohexyl sulfate;
sodium (2R,3R,4S,5R)-6-((3-dodecyloxy) propyl)amino)-2,3,4,5-tetrahydroxy-6-oxohexyl sulfate;
sodium (2R,3R,4S,5R)-6-(benzylamino)-2,3,4,5-tetrahydroxy-6-oxohexyl sulfate;
sodium (2R,3R,4S,5R)-6-((2-1H-indol-2-yl)ethyl) amino)-2,3,4,5-tetrahydroxy-6-oxohexyl sulfate;
stereoisomers thereof, salt forms thereof, or mixtures thereof.

4. The compound of claim 1 comprising a sulfate that is at least one of:
sodium (2R,3R,4S,5R)-6-(tetradecylamino)-2,3,4,5-tetrahydroxy-6-oxohexyl sulfate;
sodium (2R,3R,4S,5R)-6-(hexadecylamino)-2,3,4,5-tetrahydroxy-6-oxohexyl sulfate;
sodium (2R,3R,4S,5R)-2,3,4,5-tetrahydroxy-6-(((Z)-octadec-9-en-1-yl)amino)-6-oxohexyl sulfate;
sodium (2R,3R,4S,5R)-6-((3-(butyl(pentyl) amino) propyl)amino)-2,3,4,5-tetrahydroxy-6-oxohexyl sulfate;
sodium (2R,3R,4S,5R)-6-((3-dodecyloxy) propyl)amino)-2,3,4,5-tetrahydroxy-6-oxohexyl sulfate;
sodium (2R,3R,4S,5R)-6-(benzylamino)-2,3,4,5-tetrahydroxy-6-oxohexyl sulfate;
sodium (2R,3R,4S,5R)-6-((2-1H-indol-2-yl)ethyl) amino)-2,3,4,5-tetrahydroxy-6-oxohexyl sulfate;
and stereoisomers thereof, salt forms thereof, or mixtures thereof.

5. A detergent composition comprising a compound of claim 1.

6. The composition of claim 5, wherein said detergent composition is a form selected from the group consisting of a granular detergent, a bar-form detergent, a liquid laundry detergent, a gel detergent, a single-phase or multi-phase unit dose detergent, a detergent contained in a single-phase or multi-phase or multi-compartment water soluble pouch, a liquid hand dishwashing composition, a laundry pretreat product, a detergent contained on or in a porous substrate or nonwoven sheet, an automatic dish-washing detergent, a hard surface cleaner, a fabric softener composition, and mixtures thereof.

7. The detergent composition of claim 5, further comprising a co-surfactant.

8. The detergent composition of claim 6, wherein the co-surfactant is a zwitterionic surfactant.

9. The detergent composition of claim 6, wherein the co-surfactant comprises a betaine surfactants.

10. The detergent composition of claim 9, wherein the betaine surfactant is selected from alkyl betaines, alkyl N,N-dimethyl betaines, alkylamidobetaine, amidazoliniumbetaine, alkyl sulfobetaines, alkyl phosphobetaines, and mixtures thereof.

11. The detergent composition of claim 6, wherein the co-surfactant comprises an amine oxide surfactant.

12. The detergent composition of claim 6, wherein the co-surfactant comprises an amphiphilic alkoxylated polyalkyleneimine.

13. A cleaning composition comprising a compound of claim 1.

14. The cleaning composition of claim 13, further comprising a co-surfactant.

15. The cleaning composition of claim 14, wherein the co-surfactant is a zwitterionic surfactant.

16. The cleaning composition of claim 14, wherein the co-surfactant comprises a betaine surfactant.

17. The cleaning composition of claim 16, wherein the betaine surfactant is selected from alkyl betaines, alkyl N,N-dimethyl betaines, alkylamidobetaine, amidazoliniumbetaine, alkyl sulfobetaines, alkyl phosphobetaines, and mixtures thereof.

18. The cleaning composition of claim 14, wherein the co-surfactant comprises an amine oxide surfactant.

19. The cleaning composition of claim 14, wherein the co-surfactant comprises an amphiphilic alkoxylated polyalkyleneimine.

20. The cleaning composition of claim 14, wherein the co-surfactant comprises alkyl alkoxylated nonionic surfactants, alkylpolyglucosides, and mixtures thereof.
